SEQUENCE LISTING (5 sequences)

SEQ ID NO: 1 - protein, 21 aa, Homo sapiens
MEVGWYRPPFSRVVHLYRNGK

SEQ ID NO: 2 - protein, 9 aa, Homo sapiens
FFRDHSYQE

SEQ ID NO: 3 - protein, 18 aa, Homo sapiens
LVERGNHSKIVAGVLGLI

SEQ ID NO: 4 - protein, 25 aa, Homo sapiens
AQGTLSKIFKLGGRDSRSGSPMARR

SEQ ID NO: 5 - protein, 20 aa, Homo sapiens
ADPGSRPHLIRLFSRDAPGR

(19) United States
(12) Patent Application Publication
(10) Pub. No.: US 2017/0108514 A1
(43) Pub. Date: Apr. 20, 2017

(54) DIAGNOSIS OF MULTIPLE SCLEROSIS IN HUMAN AND ANIMAL SUBJECTS

(71) Applicant: THE REGENTS OF THE UNIVERSITY OF COLORADO, a body corporate, Denver, CO (US)

(72) Inventor: David WAGNER, Denver, CO (US)

(21) Appl. No.: 15/128,144

(22) PCT Filed: Mar. 23, 2015
(86) PCT No.: PCT/US15/22033

§ 371 Date: Sep. 22, 2016

Related U.S. Application Data
(60) Provisional application No. 61/969,214, filed on Mar. 23, 2014.
(60) Provisional application No. 61/972,114, filed on Mar. 28, 2014.

(51) Int. Cl. G01N 33/68; G01N 15/14

(52) U.S. Cl. CPC G01N 33/6896; G01N 15/14; G01N 2015/1006

(57) ABSTRACT

Cellular markers useful for methods of diagnosing multiple sclerosis (MS), relapse of MS patients and disease progression in MS patients, as well as identifying treatments for and monitoring treatment of patients with multiple sclerosis (MS). Methods of differential diagnosis of patients presenting with clinically isolated syndrome (CIS) suggestive of MS and/or Radiologically Isolated Syndrome (RIS) for presence of MS or relapse of MS, or lack thereof. Methods of treating patients having multiple sclerosis.

### GOVERNMENT INTEREST

This invention was made with government support under grant numbers DA027794 and MH076136 awarded by the National Institutes of Health. The U.S. Government has certain rights in this invention.

### TECHNICAL FIELD

The present invention relates to the identification and use of cellular markers useful in the diagnosis of multiple sclerosis (MS), relapse of MS patients and disease progression in MS patients. These markers are also useful for identifying treatments for and monitoring treatment of patients with multiple sclerosis (MS).

### BACKGROUND OF INVENTION

Multiple sclerosis (MS) is a chronic neurological and inflammatory disease of the central nervous system (CNS) in which patches of damage called plaques or lesions appear in seemingly random areas of the CNS white matter. At the site of a lesion, the nerve insulating material, myelin, is lost in a process known as demyelination. Inflammation, demyelination, oligodendrocyte death, membrane damage and axonal death all contribute to the symptoms of MS. An unpredictable disease of the central nervous system, multiple sclerosis (MS) can range from relatively benign to somewhat disabling, to devastating, as communication between the brain and other parts of the body is disrupted.

Multiple Sclerosis is a complex neurodegenerative disease. Multifocal lesions are present on brain and spinal cord, predominantly in white matter although gray matter infiltrates are detected (Lucchinetti et al., 2011). The disease has multifactorial, yet unresolved etiology with genetic, immunologic and environmental factors each presumed contributors. The most common disease course is relapsing/remitting multiple sclerosis (RRMS) accounting for 85% of cases with more than 50% of those eventually becoming secondary progressive (SPMS), a more severe course of disease (Hemmer et al., 2002). RRMS patients have CNS lesions characterized by edema and demyelination resulting from an inflammatory process (Miller et al., 1998); in 82% of lesions, CD3+ T cell infiltrates were detected (Lucchinetti et al., 2011). Findings suggest that inflammation mediated by immune activation is prevalent early, an attribute that is consistent with progressive autoimmunity.

Diagnosis of MS is relatively complicated. A wide range of symptoms including: blurred or tunnel vision, general weakness, numbness, prickling pain, muscle spasm(s), tremors, dizziness, headaches, paraparesis, memory loss, speech impediment, attention deficit, decreased concentration etc., are associated with the disease. However these symptoms are likewise associated with other neurologic disorders and diverse diseases such as vascular diseases, infectious diseases, vitamin B-12 deficiency, hypo-thyroidism and even brain tumors. In order to diagnose MS, disease criteria were defined by what came to be known as the McDonald Criteria, which were revised in 2005 (Polman et al., 2005) and again in 2010 (Polman et al., 2011). The revised criteria require clinical and para-clinical laboratory assessments e.g. composite symptoms with MRI and spinal taps, etc., with demonstrated dissemination of lesions (MRI) in space (DIS) and time (DIT) (Polman et al., 2011). The new criteria however do not advance understanding of the autoimmune contributors to MS. Even with the changes to the McDonald criteria it “remains imperative that alternative diagnoses are considered and excluded” (Polman et al., 2011). The International Panel on Diagnosis of MS stressed that the McDonald criteria “should only be applied in those patients who present with a typical clinically isolated syndrome (CIS) suggestive of MS or symptoms consistent with a CNS inflammatory demyelinating disease” (Polman et al., 2011). CIS can include optic neuritis, brainstem or spinal cord syndromes. While these often are the first clinical presentations of MS, not all CIS patients develop MS, for example in one longitudinal (20 year) study, only 63% of CIS patients advanced to clinically defined MS (CDMS) over that time period (Fisniku et al., 2008). A CIS diagnosis requires symptoms and an abnormal but static MRI scan; thus no DIS or DIT. Another issue is Radiologically Isolated Syndrome (RIS) which involves laboratory findings typical of demyelination but in healthy patients that are asymptomatic or present non-specific symptoms (e.g., headache, dizziness) (Miller et al., 2012). Longitudinal studies report that about 30-40% of these patients eventually progress to clinically defined MS.

The revised McDonald Criteria reaffirmed that positive cerebral-spinal fluid (CSF) findings e.g., the presence of oligoclonal (Immunoglobulin) bands, which is at present the only laboratory test that focuses on the autoimmune component of MS, are important “to support the inflammatory demyelinating nature of the underlying condition, to evaluate alternative diagnoses, and to predict CDMS” (Polman et al., 2011). Given the highly invasive and restricted nature of this single test, detecting oligoclonal bands is often not specific, i.e. non-autoimmune individuals can have oligoclonal bands and autoimmune individuals may not demonstrate bands. Thus, a blood test that identifies biomarkers for the autoimmune component(s) of MS would be highly desirable. There is also a need for improved methods to diagnose relapse or disease progression in subjects that are being treated for multiple sclerosis.

T cells have been identified that defy standard definitional criteria by expressing the antigen presenting cell (APC) associated molecule CD40 and thus have been termed Th40 cells (Siebert et al., 2008; Waid et al., 2008; Waid et al., 2004; Waid et al., 2007). Th40 cells were expanded as a percentage of peripheral blood lymphocytes and in terms of absolute numbers in autoimmune diabetes including both the mouse model of type 1 diabetes (T1D) (Waid et al., 2004; Waid et al., 2007) and in human studies (Siebert et al., 2008; Waid et al., 2007). Th40 cells rapidly and consistently transfer T1D to NOD.scid recipients. In human studies when T1D patients are compared to non-autoimmune controls, Th40 cells are significantly (p<0.00001) expanded in peripheral blood of both new onset and long term T1D patients and Th40 cells respond to diabetes associated antigens producing Th1, pro-inflammatory, cytokines (Waid et al., 2007).

CD40 is a critical player in several autoimmune diseases including, diabetes, arthritis, colitis, EAE (the mouse model for MS) (Girvin et al., 2002) and in human MS (Benveniste et al., 2004; Giuliani et al., 2005). CD40 as a dominant player in so many diverse autoimmune diseases suggests that it constitutes a critical and early phase autoimmune inflammation marker. The focus of previous CD40 investigations has been almost exclusively in CD40 as an antigen presenting cell modulator. CD40 is also expressed on T cells (Carter et al., 2012b; Vaitaitis et al., 2010; Vaitaitis et al., 2013; Vaitaitis et al., 2003; Vaitaitis and Wagner, 2008; Vaitaitis and Wagner, 2010; Vaitaitis and Wagner, 2012; Vaitaitis and Wagner Jr., 2013; Wagner et al., 2002; Waid et al., 2008; Waid et al., 2004; Waid et al., 2007).

### SUMMARY OF INVENTION

The present invention provides a method to diagnose a subject as having multiple sclerosis, comprising:

determining the percentage of Th40 cells in a sample isolated from the subject,

comparing the percentage of Th40 cells to a control sample or a standard value

diagnosing multiple sclerosis in the subject, wherein an increase in the percentage of Th40 cells in the sample from the subject relative to the control sample or standard value is indicative of multiple sclerosis in the subject.

The invention further provides a method to diagnose relapse or disease progression in a subject having multiple sclerosis that is being treated for multiple sclerosis, comprising:

determining the percentage of Th40 cells in a sample isolated from the subject,

comparing the percentage of Th40 cells to a control sample or a standard value, and

diagnosing relapse or disease progression, wherein the percentage of Th40 cells in the sample from the subject indicates the subject is in relapse or has disease progression.

The invention further provides a method to identify a composition useful for the treatment of multiple sclerosis in a subject, comprising: administering a test composition to the subject, evaluating whether administration of the test composition causes a transient change in the percentage of Th40 cells in a sample isolated from the subject, wherein a transient decrease in the percentage of Th40 cells in samples from the subject over about 1-6 days indicates the test composition is useful for treatment of multiple sclerosis.

The invention also provides a method to identify a dose of a composition, useful for the treatment of multiple sclerosis in a subject, to elicit a desired magnitude of response, comprising:

administering the composition to MS subjects at different doses,

evaluating the change in the percentage of Th40 cells in a sample isolated from the MS subjects at the different doses of the composition; and

identifying a dose of the composition that elicits the desired magnitude of decrease in the percentage of Th40 cells in samples from the subject over about 1-6 days indicates the test composition is useful for treatment of multiple sclerosis.

The invention further provides a method to monitor treatment of multiple sclerosis in a subject. This methods includes administering a therapeutic composition to the subject, and evaluating whether administration of the therapeutic composition causes a transient decrease in the percentage of Th40 cells in a sample isolated from the MS subjects following administration of the composition; wherein a transient decrease in the percentage of Th40 cells in the samples from the subject indicates efficacious treatment of multiple sclerosis in the subject.

The invention also provides a method to confirm or rule out diagnosis of multiple sclerosis (MS) in a subject presenting with typical clinically isolated syndrome (CIS) suggestive of MS or symptoms consistent with a CNS inflammatory demyelinating disease. The method includes determining the percentage of Th40 cells in a sample isolated from a subject presenting with these clinical syndromes or symptoms, comparing the percentage of Th40 cells to a control sample or a standard value, and, diagnosing multiple sclerosis in the subject having an increase in the percentage of Th40 cells in the sample from the subject relative to the control sample or standard value or, ruling out multiple sclerosis in the subject having no increase or a decrease in the percentage of Th40 cells in the sample from the subject relative to the control sample or standard value.

A similar method of the invention may be used to confirm or rule out diagnosis of multiple sclerosis (MS) in a subject presenting with Radiologically Isolated Syndrome (RIS) suggestive of MS. This method includes determining the percentage of Th40 cells in a sample isolated from the subject presenting with RIS, comparing the percentage of Th40 cells to a control sample or a standard value, and, diagnosing multiple sclerosis in the subject having an increase in the percentage of Th40 cells in the sample from the subject relative to the control sample or standard value, or ruling out multiple sclerosis in the subject having no increase or a decrease in the percentage of Th40 cells in the sample from the subject relative to the control sample or standard value.

Another method of the invention may be used to confirm or rule out the presence of relapsing/remitting multiple sclerosis (RRMS) in a subject. The method includes determining the percentage of Th40 cells in a sample isolated from a subject presenting with a clinically isolated syndrome (CIS) suggestive of MS, and/or Radiologically Isolated Syndrome (RIS) suggestive of MS, comparing the percentage of Th40 cells to a control sample or a standard value, and diagnosing RRMS in the subject having an increase in the percentage of Th40 cells in the sample from the subject relative to the control sample or standard value or, ruling out a relapse of MS in the subject having no increase or a decrease in the percentage of Th40 cells in the sample from the subject relative to the control sample or standard value.

In some embodiments of these methods, the control sample is a sample from at least one subject known not to have multiple sclerosis.

In some embodiments of these methods, the control sample is a sample from at least one subject known to have multiple sclerosis.

In some embodiments of these methods, the control sample is a sample from at least one subject known to have relapsing-remitting multiple sclerosis (RRMS).

In some embodiments of these methods, the control sample is a sample from at least one subject known to have primary-progressive multiple sclerosis (PPMS).

In some embodiments of these methods, the control sample is a sample from at least one subject known to have secondary-progressive multiple sclerosis (SPMS).

In some embodiments of these methods, the control sample is a sample from at least one subject known to have Clinically isolated syndrome (CIS).

In some embodiments of these methods, the control sample is a sample from at least one subject known to have radiologically isolated syndrome (RIS).

In some embodiments of these methods, the control sample is a baseline sample obtained from the subject at an earlier date.

In some embodiments of these methods, the control sample is a sample obtained from at least one Type-1 Diabetes (T1D) patient.

In some embodiments of these methods, the control sample is a sample obtained from at least one Type-2 Diabetes (T2D) patient.

In some embodiments of these methods, the sample is whole blood, plasma, serum, or a subfraction of whole blood.

In some embodiments of these methods, the method further comprises determining the percentage of cells expressing additional markers selected from CD4, CD40, CD8, CD25, CD45, TCRV8.3+, CXCR3, and CCR5.

In some embodiments of these methods, the method comprises staining with a labeled antibody that specifically recognizes a protein selected from CD40, CD4, CD8, CD25 and CD45 and analyzing the stained cells by flow cytometry to determine the percentage of stained cells in the sample.

In some embodiments of these methods, the subject is being treated by the administration of an interferon.

In some embodiments of these methods, the subject is selected from a human, a non-human primate, and rodents. In some embodiments wherein the subject is human, the subject can be a healthy subject, a naïve subject, or a subject on an established treatment of at least one of Aubagio (teriflunomide), betaseron (interferon-b (type 1)), Avonex (Interferon-b-1b), Rebif (interferon-beta-1a), Copaxone (Glatiramer Acetate), Tysabri (Natalizumab), Novantrone (mitoxantrone), Gilenya (fingolimod), Tecfidera (dimethyl fumarate), and/or Lemtrada.

Another embodiment of the invention is a method of predicting or assessing the level of severity of multiple sclerosis in a patient diagnosed with multiple sclerosis. The method includes determining the percentage of Th40 cells in a sample from the patient and comparing the percentage of Th40 cells in the sample to the percentage of Th40 cells in a control sample or a standard value. An increase in the percentage of Th40 cells in the sample relative to the ratio in the control sample or standard value is indicative of a more severe form of multiple sclerosis or relapsing stage of multiple sclerosis or progressing form of multiple sclerosis in the patient. A decrease in the percentage of Th40 cells in the sample relative to the ratio in the control sample or standard value is indicative of a less severe form of multiple sclerosis or remitting stage of multiple sclerosis or stable form of multiple sclerosis or non-progressing form of multiple sclerosis in the patient.

Another embodiment of the invention is a method for identifying and treating a patient for multiple sclerosis. This method includes detecting the percentage of Th40 cells in T-cells in a blood sample from the patient and treating the patient with an elevated percentage of Th40 cells relative to a percentage of Th40 cells in a blood sample from a normal control sample with a drug in an amount effective to decrease the percentage of Th40 cells in the patient's blood.

The drugs that may be administered to the patient may include any one of Aubagio (teriflunomide), betaseron (interferon-b (type 1)), Avonex (Interferon-b-1b), Rebif (interferon-beta-1a), Copaxone (Glatiramer Acetate), Tysabri (Natalizumab), Novantrone (mitoxantrone), Gilenya (fingolimod), Tecfidera (dimethyl fumarate), Lemtrada, or combinations of these drugs.

Additional drugs that may be administered to the patient identified as having an elevated percentage of Th40 cells may include a drug selected from a corticosteroid, an interferon, Glatiramer acetate (Copaxone), Fingolimod (Gilenya), Natalizumab (Tysabri), Mitoxantrone, Teriflunomide (Aubagio), Dalfampridine (Ampyra), baclofen (Lioresal) and tizanidine (Zanaflex), amantadine, Aubagio (teriflunomide), Avonex (interferon beta-1a), Betaseron (interferon beta-1b), Extavia (interferon beta-1b), Gilenya (fingolimod), Novantrone (mitoxantrone), Rebif (interferon beta-1a), Tecfidera (dimethyl fumarate), Tysabri (natalizumab), Botox (onabotulinumtoxin A), DDAVP Nasal Spray (desmopressin), Detrol (tolterodine), Ditropan (oxybutynin), Ditropan XL, Enablex (darifenacin), Flomax (tamsulosin), Hytrin (terazosin), Minipress (prazosin), Oxytrol (oxybutynin), Pro-Banthine (propantheline), Sanctura (trospium chloride), Tofranil (imipramine), Vesicare (solifenacin succinate), Bactrim (sulfamethoxazole), Cipro (ciprofloxacin), Macrodantim (nitrofurantoin), Hiprex (methenamine), Pyridium (phenazopyridine), Colace (docusate), Dulcolax (bisacodyl), Enemeez (docusate stool softener laxative), Fleet Enema (sodium phosphate), Mineral Oil, Metamucil (psyllium hydrophilic musilloid), Phillips Milk of Magnesia (magnesium hydroxide), Sani-Supp suppository (gylcerin), Cymbalta (duloxetine hydrochloride), Effexor (velafaxine), Paxil (paroxetine), Prozac (fluoxetine), Wellbutrin (bupropion), Zoloft (sertraline), Antivert (meclizine), Nuedexta (dextromethorphan+quinidine), Provigil (modafinil), Atarax (hydroxyzine), Dilantin (phenytoin), Elavil (amitriptyline), Klonopin (clonazepam), Neurontin (gabapentin), Aventyl (nortriptyline), Tegetrol (carbamazepine), Cialis (tadalafil), Levitra (vardenafil), Papaverine, MUSE (alprostadil), Prostin VR (alprostadil), Viagra (sildenafil), Dantrium (dantrolene), Gablofen (baclofen [intrathecal]), Klonopin (clonazepam), Valium (diazepam), Zanaflex (tizanidine), Laniazid-Nydrazid (isoniazid), Ampyra (dalfamridine), Idebenone, Rituximab, and Plegridy (peginterferon beta-1a).

In these methods, the percentage of Th40 cells in the sample may be assessed by staining with a labeled antibody that specifically recognizes a protein selected from CD40, CD4, CD8, CD25 and CD45 and analyzing the stained cells by flow cytometry to determine the percentage of stained cells in the sample.

### BRIEF DESCRIPTION OF DRAWINGS

FIG. 1 shows that Th40 cell percentage is expanded in MS. Blood samples were taken from diagnosed MS, including RRMS, PPMS and SPMS subjects and non-autoimmune controls. Blood samples likewise were taken from T1D and T2D subjects. Samples were analyzed in a blinded study. FIG. 1A shows dot plots were generated following CD4, CD3 and CD40 staining Levels of CD4 (x-axis) versus CD40 (y-axis) are shown. Gates were set from isotype controls for each group. Gated CD4⁺CD40⁺ cells demonstrate CD3 expression (histogram, gray line) above isotype control (red line). FIG. 1B shows the percent of CD40⁺ cells within the CD4⁺ compartment from 168 MS, 98 T1D, 112 non-autoimmune control and 62 T2D subjects. FIG. 1C shows a comparison of the percent of CD40⁺ cells within the CD4⁺ compartment as a function of the subject's age. FIG. 1D shows a comparison of RRMS (48 randomly chosen subjects), PPMS (6 subjects) and SPMS (8 subjects). Statistics were non-paired t-tests determined using the GraphPad Prism program and ANOVA, Tukey test.

FIG. 2 shows Treg levels in MS subjects demonstrating that CD40+ T cells occur both as CD4low and CD4hi T cells in MS.MS patient samples (n=48) that had been stained with exactly the same stain sets of antibodies were compared to control subjects (n=8) for CD4 expression level (CD4hi or CD4lo) as well as CD40 intensity within the CD4hi and CD4lo populations. Due to the large range in Th40 levels, MS samples were divided into groups of high (MS N 70%), intermediate (50% N MS b 70%), and low (MD b 50%) Th40 levels. Cells were gated on live cells then CD4 and CD40 was gated based on isotype controls. FIG. 2A shows Th40 levels (CD4+CD40+ of total CD4+ T cells) in MS samples compared to control. Red box—MS N 70%; Black box—50% N MS b 70%; Blue box—MD b 50%. FIG. 2B shows dot plots of CD4 versus CD40 stains representative of the three Th40 level groups in MS. Red—isotype control; Blue CD4/CD40 stained. Further gates were set around CD4lo and CD4hi populations for analysis in D and E. FIG. 2C shows Th40 levels within the three groups compared to each other and control. FIG. 2D shows the percent CD4lo and CD4hi within each of the MS groups and control. FIG. 2E CD40 mean fluorescence intensity (MFI) within the CD4lo and CD4hi gated populations in the different MS groups and control. Statistical analysis was done by one-way ANOVA followed by a Bonferroni post-test; ****—p<0.0001, ***p<0.001, **—p<0.01, *—p between 0.01 and 0.05.

FIG. 3 shows Treg levels in MS subjects. Percent Tregs was defined as CD4+CD25hiFOXP3+ cells in peripheral blood of RRMS, non-autoimmune control and T1D subjects. Subjects were age and gender matched as in FIG. 1. MS and T1D patients had well established disease courses, diagnosed for greater than 5 years. Cells were stained with CD4, CD25 and intra-cellular FOXP3. Tregs were designated as CD25hi, CD4+, and FOXP3+. FIG. 3A shows the percentage of Tregs from individual subjects was compared. Each symbol represents an individual patient. FIG. 3B shows the ratio of Th40 cell to Tregs within each individual was determined. Bar graphs represent 16 individuals from each group. FIG. 3C shows the Treg level analysis based on age groups (b35 years old; 35-b50 years old: 50 and older). FIG. 3D shows the Th40 to Treg ratio analysis based on age groups (b35 years old; 35-b50 years old: 50 and older). Statistics were non-paired t-tests and Tukey ANOVA analysis determined using the GraphPad Prism program.

FIG. 4 shows the Th40 cell percentages relative to HLA haplotype. FIG. 4A shows HLA-DR haplotypes were determined in a cohort of 48 RRMS subjects. Each HLA allele was considered individually. FIG. 4B shows Th40 cell percentages were determined within each haplotype. Although each haplotype showed a wide range in Th40 percentage, there were no statistical differences in means of Th40 cell percentages relative to any HLA haplotype within the MS cohort. FIG. 4C shows HLA-DQ haplotypes were determined in the 48 RRMS subjects. FIG. 4D shows Th40 cell percentages were not significantly different relative to any given HLA-DQ allele. Statistics were non-paired t-tests and ANOVA determined using the GraphPad Prism program.

FIG. 5 shows the limited T cell clonality in Th40 cells from a RRMS cohort. A cohort of 48 RRMS subjects was examined for potential T cell clonality. Purified peripheral T cells were stained for CD4, CD40, CD45RO and TCR Vα8.3. CD4 and CD40 expressions were determined above isotype control (shown). The percent Vα8.3+ cells above isotype controls (grey line) were determined in CD4+CD40+ and CD4+CD40− populations. 16 out of 48 subjects demonstrated an increase in Vα8.3+ T cells only within the CD4+CD40+ population. Gated Vα8.3+ cells were examined for CD45RO, memory phenotype.

FIG. 6 shows the antigen response of Th40 cells from MS subjects. Blood collected from 20 RRMS subjects was separated into APC and T cell fractions. APC were exposed to peptide antigens then fixed by U.V. Strata-link exposure. T cells were CFSE labeled. T cells then were exposed to autologous APC/Ag for 7 days and total proliferation above negative controls was determined. Percent response was determined by subtracting background proliferation from antigen induced proliferation. Data were analyzed relative to HLA-DR haplotype. FIG. 6A shows data from patients with DR2 at either allele with an open second allele; FIG. 6B shows DR6 at either allele with open second allele; FIG. 6C shows DR6 at both alleles; FIG. 6D shows DR2 at one allele and DR6 at the second allele; FIG. 6E shows DR4 at either allele with an open second allele; FIG. 6F shows DR4 at one allele and DR2 at the second allele; FIG. 6G shows DR4 at one allele and DR6 at the other allele. Statistics were unpaired t-tests or Mann-Whitney ANOVA analysis performed by the GraphPad Prism program. Statistically significant differences are noted.

FIG. 7 shows CD45R0memory T cell marker in Th40 and CD4+CD40− T cells. T cells were selected by depleting HLA+ cells from isolated lymphocytes then stained for CD4, CD40 and CD45RO in (FIG. 7A) MS subjects, 48 total subjects and age matched (FIG. 7B) non-autoimmune control, 48 total subjects. Dot plots show representative CD4 versus CD40 levels with gates set from isotype controls. Levels of CD45RO+ cells were determined (black line) above isotype controls (red line) in Th40 cells, top histogram of each set, and in CD4+CD40− cells, bottom histogram in each set. The CD45RO+ population in Th40 cells from MS is statistically (p<0.001) different from CD45RO+CD4+CD40− cells and from both populations of cells from non-autoimmune controls. Mean percentages are shown +/−SEM. Statistics were ANOVA, Tukey comparison, using Graph Pad Prism program.

FIG. 8 shows memory phenotype Th40 cells from MS patients produce both IL-17 and IFNγ. Total blood lymphocytes were stained and gated for CD4 and CD45RO expression to delineate memory cells. FIG. 8A shows that within that population, Th40 cells were assayed for IFNγ and IL-17 production by intra-cellular staining Levels of IL-17 and IFNγ were determined above isotype controls. Cells were examined immediately ex vivo. FIG. 8B is a graph representing levels of IFNγ only, IL-17 only and IFNγ/IL17 double positive cells from 11 RRMS subjects. Statistics were done by ANOVA, Tukey comparison, using the Graph Pad Prism program.

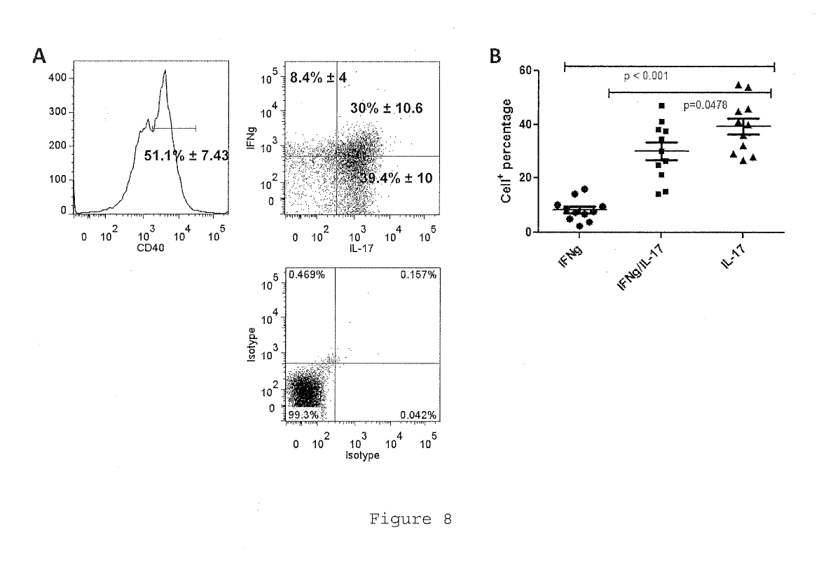

FIG. 9 shows the HLA-DR haplotypes were determined in a cohort of 48 RRMS subjects and determined in 48 age-matched non-autoimmune controls. Th40 cell percentages were determined within the haplotypes listed for MS versus control subjects. In each haplotype direct comparison, MS subjects Th40 cell levels were significantly (p<0.001) greater than Th40 cell levels from haplotype matched non-autoimmune controls.

### DESCRIPTION OF ILLUSTRATIVE EMBODIMENTS

The present invention includes methods to diagnose a subject as having multiple sclerosis, methods to diagnose relapse or disease progression in a subject having multiple sclerosis to identify a composition useful for the treatment of multiple sclerosis, methods to identify an appropriate dose of a composition useful for the treatment of multiple sclerosis, methods to monitor treatment of a subject for multiple sclerosis.

These methods are generally accomplished by phenotyping subjects, e.g., measuring for the presence, absence, increase, decrease or other changes in particular cellular markers and/or characteristics of these specific cells in response to administration of compositions in accordance with the present invention. These biomarkers and/or characteristics can be detected by any method for analysis of amount or expression of these markers, including, without limitation, cytometry, immunoassay, mass spectrometry, and methods for quantifying proteins and nucleic acids.

Methods of the present invention are based on a study that addressed the impact of Th40 cells, a pathogenic effector subset of helper T cells, in MS. MS patients including relapsing/remitting MS (RRMS), secondary progressive MS (SPMS) and primary progressive MS (PPMS) were examined for Th40 cell levels in peripheral blood and the levels were significantly (p<0.0001) elevated compared to controls including healthy non-autoimmune subjects, another non-autoimmune chronic disease, and autoimmune type 1 diabetes. Surprisingly, classically identified Tregs were elevated above controls but the Th40/Treg ratio still predicted autoimmunity. The cohort displayed a wide range of HLA haplotypes including the GWAS identified predictive HLA-DRB1*1501 (DR2). However half the subjects did not carry DR2 and regardless of HLA haplotype, Th40 cells were expanded during disease. In RRMS Th40 cells demonstrated a limited TCR clonality. Mechanistically, Th40 cells demonstrated a wide array of response to CNS-associated self-antigens that was dependent upon HLA haplotype. Th40 cells were predominantly memory phenotype producing IL-17 and IFNγ with a significant portion producing both inflammatory cytokines simultaneously, suggesting an intermediary between Th1 and Th17 phenotypes.

Statistical comparisons were made in the study with appropriate linear mixed effect models and variables showing differences ranked by univariate p-value levels in consideration of multiple comparisons with tested variables. The markers indicate drug-target interactions and are useful for, among others, diagnosing, evaluating dosing, evaluating test compounds/compositions, and monitoring treatment. Between cohort comparisons reflect differences that relate to, among others, disease pathophysiology, disease progression and efficacy of treatment.

A CD4+ T cell subset has been identified that expresses the CD40 receptor, termed Th40 cells. The inventors have shown that those T cells directly impact autoimmune pathogenesis (Carter et al., 2012a; Siebert et al., 2008; Vaitaitis and Wagner Jr., 2010; Vaitaitis et al., 2010; Vaitaitis et al., 2003; Vaitaitis and Wagner, 2008; Vaitaitis and Wagner, 2010; Vaitaitis and Wagner, 2012; Wagner et al., 2002; Waid et al., 2008; Waid et al., 2004; Waid et al., 2007). These Th40 cells passively transfer type 1 diabetes in mouse models but importantly are involved in human disease (Siebert et al., 2008; Waid et al., 2007). As autoimmunity develops, Th40 cells increase in percentage and increase in number systemically (Vaitaitis et al., 2003; Wagner et al., 2002; Waid et al., 2008; Waid et al., 2004). We determined that Th40 cell levels do not wax and wane throughout autoimmunity as other ‘activated’ cells e.g. HLA+, CD30+, CD25+, CD69+ or CD154+, do. In longitudinal studies in human T1D, Th40 cell levels are as high in subjects who were diagnosed greater than 45 years previously as to those who were diagnosed 2 weeks prior (Siebert et al., 2008; Waid et al., 2007). In human T1D studies the Th40 occurrence remains relatively close to the disease associated mean, however in MS subjects, Th40 cell levels varied a great deal relative to the mean. The majority of subjects examined were relapsing/remitting multiple sclerosis (RRMS), thus the variance in Th40 cell levels may reflect the relapsing/remitting nature of the disease. In fact RRMS subjects demonstrate wide Th40 variation while secondary progressive MS (SPMS) and primary progressive MS (PPMS) have less variation.

These studies showed that a cohort of RRMS patients demonstrates TCR clonality within the Th40 cell populations and Th40 cells are responsive to MS associated antigens. Importantly Th40 cells are responsive to several different CNS antigens, suggesting a broad repertoire array localized within those T cells. Because of this diversity, Th40 cells would be primed to prolong pathogenesis and as shown here, likely through production of the inflammatory cytokines IFNγ and IL-17.

Alternative approaches to tolerance induction in MS now include multiple antigen treatments. In a human trial study of MS patients, an infusion of autologous blood mononuclear cells that were chemically coupled to 7 different MS associated peptides including MOG35 and MBP83, which were used in this study, demonstrated a decrease in antigen specific T cell responses (Lutterotti et al., 2013). This is encouraging but autoimmunity will likely require a multi-faceted treatment approach. By identifying a cellular marker, in this case CD40, which is associated with long-term pathogenic T cells and specifically respond to MOG35 and MBP83, among other antigens, a new approach to tolerance emerges; inducing anergy directly through CD40 blockade. Previous attempts at universal pathogenic T cell control have focused on controlling CD28 or T cell activation steps mediated by ZAP70. These attempts have not been successful. By considering the actions of CD40, specifically directed towards pathogenic T cells, controlling autoimmune inflammation may be possible.

According to the present invention, the term “multiple sclerosis” (MS) is used to describe the art-recognized disease characterized by inflammation, demyelination, oligodendrocyte death, membrane damage and axonal death. MS can be more particularly categorized as either relapsing/remitting MS (observed in 85-90% of patients) or progressive MS. In some embodiments, MS can be characterized as one of four main varieties as defined in an international survey of neurologists (Lublin and Reingold, 1996, Neurology 46(4):907-11), which are namely, relapsing/remitting MS, secondary progressive MS, progressive/relapsing MS, or primary progressive MS (PPMS).

As used herein, the terms “patient”, “a subject who has MS”, “a patient who has MS”, “an MS subject”, “an MS patient”, and similar phrases, are intended to refer to subjects who have been diagnosed with MS. The terms “Healthy subject”, “non-MS subject”, “a subject who does not have MS”, “a patient who does not have MS”, and similar phrases, are intended to refer to a subject who has not been diagnosed with MS. A Healthy subject has no other acute systemic disease. The term “naïve”, and “naïve subject” refers to subjects with MS previously naïve to drug therapy.

As used herein, the term “sample” or “biological sample” includes a sample of any cell type or from any tissue or body fluid, body fluids including, but not limited to: cerebrospinal fluid (CSF), serum, plasma, blood, or fluid from any suitable tissue. In a preferred embodiment, the biological sample is blood or any component of blood (e.g., serum, plasma, etc.).

As used herein, “interferon”, “interferons”, “interferon treatment” include, for example, type I interferons (alpha, beta, omega, epsilon and kappa), type II interferons (gamma) and interferon lambda, and additional naturally-occurring subtypes and/or isoforms of interferon, produced by different cell types. Preferred compositions of the present invention include interferon beta variants, homologues, and fragments which have substantially similar biological activity as interferon beta 1a as measured by, for example, the specific activity of the molecule compared to the antiviral activity of the reference standard of recombinant human Interferon beta or Interferon beta 1a. Additionally, these terms include homologues of interferon with substantially similar biological activity are encompassed by the present invention. As used herein, the term “homologue” is used to refer to a polypeptide which differs from a naturally-occurring polypeptide by one or more minor modifications or mutations to the naturally-occurring polypeptide, but which maintains the overall basic protein and side chain structure of the naturally-occurring form (i.e., such that the homologue is identifiable as being related to the wild-type polypeptide). Such changes include, but are not limited to: changes in one or a few amino acid side chains; changes to one or a few amino acids, including deletions (e.g., a truncated version of the protein or peptide) insertions and/or substitutions; changes in stereochemistry of one or a few atoms; and/or minor derivatizations, including but not limited to: PEGylation (polyethylene glycol modifications), methylation, farnesylation, geranyl geranylation, glycosylation, carboxymethylation, phosphorylation, acetylation, myristoylation, prenylation, palmitation, and/or amidation. A homologue can have either enhanced, decreased, or substantially similar properties as compared to the naturally occurring polypeptide. Homologues can include synthetically produced homologues, naturally occurring allelic variants of a given protein or domain, or homologous sequences from organisms other than the organism from which the reference polypeptide was derived. Test compositions to be screened also include known organic compounds such as peptides (e.g., products of peptide libraries), oligonucleotides, carbohydrates, synthetic organic molecules (e.g., products of chemical combinatorial libraries), and antibodies. Compounds may also be identified using rational drug design relying on the structure of the product of a gene or polynucleotide. Such methods are known to those of skill in the art and involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

Other drugs that may be used in the methods of the invention include:

Aubagio (teriflunomide), which is an orally available pyrimidine synthesis inhibitor, that is immune suppressive. This drug reduced relapses in a phase I study, but has moderate potential for hepatotoxicity.

Interferons, including: Betaseron: interferon-b (type 1), Avonex: (Interferon-b-1b), and Rebif (interferon-beta-1a).

Copaxone (Glatiramer acetate), which is a four-amino acid peptide that look similar to myelin. Therapy with this drug typically entails daily injections, and is thought to decrease response to myelin and increase the number of regulatory T cells. Clinical reports describe a decreasing frequency and severity of attacks using Copaxone treatment, but there are no reports of changes in MRI lesions at this time.

Tysabri (Natalizumab) is a humanized monoclonal antibody that blocks VLA4 receptors on lymphocytes allowing them to enter the brain and spinal cord. The drug is typically administered by IV infusion once every four weeks. Tysabri therapy has been reported to slow progression, and have some positive effects on MRI. These reports indicate that subjects may experience a two-thirds reduction in relapse rate. Currently therapy with this drug is restricted to approved infusion centers.

Novantrone (mitoxantrone) is a chemotherapeutic agent that slows progression and lessens relapses of MS. this drug is a T cell/B cell immunosuppressive that can only be used over a limited time period. It is administered intravenously once every three months for two years.

Gilenya (fingolimod) is a sphingosine 1-phosphate receptor modulator, thought to suppress lymphocytes in lymph nodes to prevent crossing the blood brain barrier. The drug is extremely immune-suppressive and is contraindicated for patients with cardiac disorders.

Tecfidera (dimethyl fumarate) is an orally available drug tought to inhibit immune cells. This drug may have anti-oxidant properties, but the precise mechanism of action is unknown. The drug produces some reduction in the average annual number of relapses but disability progression was not reduced significantly.

Lemtrada (alemtuzumab) is a monoclonal antibody that binds to CD52, a protein present on the surface of mature lymphocytes, but not on the stem cells from which these lymphocytes are derived. The CD52-bearing lymphocytes are targeted for destruction. Unfortunately, this drug significantly increases the risk for opportunistic infections, in particular, reactivation of cytomegalovirus.

Other compositions of the present invention include mimetics. As used herein, a mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

Candidate test compositions identified or designed by the methods of the invention can be synthesized using techniques known in the art, and depending on the type of compound. Synthesis techniques for the production of non-protein compounds, including organic and inorganic compounds are well known in the art. For example, for smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture. A compound that is a protein or peptide can also be produced using recombinant DNA technology and methods standard in the art, particularly if larger quantities of a protein are desired.

As used herein, the terms “test composition”, “test compound”, “putative inhibitory compound” or “putative regulatory compound” refer to compositions having an unknown or previously unappreciated regulatory activity in a particular process. As such, the term “identify” with regard to methods to identify compounds is intended to include all compositions, the usefulness of which as a regulatory compound for the purposes of regulating the expression or activity of a target biomarker or otherwise regulating some activity that may be useful in the study or treatment of MS is determined by a method of the present invention.

The terms “cellular marker”, “biomarker” or “marker”, as used herein, can refer to a cell, particularly a blood cell, a ratio of particular cells, a cell-associated polypeptide or protein, a soluble polypeptide or metabolite described herein or to a polynucleotide (including a gene) that encodes a polypeptide identified by the invention. In addition, the terms “biomarker” or “marker” can be generally used to refer to any portion or component of such a cell, portion or component indicating the ratio of particular cells, cell-associated polypeptides, soluble polypeptides or polynucleotides that can identify or correlate with the cell, ratio of particular cells, full-length polypeptide or polynucleotide, for example, in an assay of the invention. Biomarkers also include any components or portions of cells, precursors and successors of polypeptides and polynucleotides of the invention, as well as polypeptides and polynucleotides substantially homologous to polypeptides and polynucleotides of the invention. Accordingly, a biomarker useful in the present invention is any cell, cell ratio, polynucleotide, polypeptide or metabolite, the expression or occurrence of which is regulated (up or down) in a subject with a condition (e.g., MS) as compared to a normal control.

Of the cells analyzed, the present inventors have identified multiple biomarkers, (i) the expression of which are regulated differentially in subjects with MS as compared to subjects without MS, (ii) that cause short-term changes upon drug therapy, and (iv) that cause longer-term changes upon drug therapy among the different cohort populations. More particularly, the biomarkers are selectively upregulated in the blood for relapsing/remitting MS (RRMS), secondary progressive MS (SPMS) and primary progressive MS (PPMS) subjects.

As used herein, compositions and/or biomarkers comprising peptides can be substantially homologous or homologues to another composition or to the biomarker, respectively. Two polypeptides are “substantially homologous” or “homologues” when there is at least 70% homology, at least 80% homology, at least 90% homology, at least 95% homology or at least 99% homology between their amino acid sequences, or when polynucleotides encoding the polypeptides are capable of forming a stable duplex with each other. As used herein, unless otherwise specified, reference to a percent (%) identity refers to an evaluation of homology which is performed using: (1) a BLAST 2.0 Basic BLAST homology search using blastp for amino acid searches, blastn for nucleic acid searches, and blastX for nucleic acid searches and searches of translated amino acids in all 6 open reading frames, all with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389, incorporated herein by reference in its entirety); (2) a BLAST 2 alignment (using the parameters described below); (3) and/or PSI-BLAST with the standard default parameters (Position-Specific Iterated BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

As used herein, a “fragment” of a polypeptide refers to a single or a plurality of amino acid residues comprising an amino acid sequence that has at least 5 contiguous amino acid residues, at least 10 contiguous amino acid residues, at least 20 contiguous amino acid residues, at least 30 contiguous amino acid residues, at least 40 contiguous amino acid residues, at least 50 contiguous amino acid residues, at least 60 contiguous amino acid residues, at least 70 contiguous amino acid residues, at least 80 contiguous amino acid residues, at least 90 contiguous amino acid residues, or at least 100 contiguous amino acid residues of a sequence of the polypeptide, or any number of residues between 5 and 100, in whole number increments.

As used herein, a polypeptide is referred to as “isolated” when it has been removed from its natural milieu (i.e., that has been subject to human manipulation), and can include purified polypeptides, partially purified polypeptides, synthetically produced polypeptides, and recombinantly produced polypeptides, for example. As such, “isolated” does not reflect the extent to which the polypeptide has been purified.

In some embodiments, a biomarker of the invention is a member of a biological pathway. As used herein, the term “precursor” or “successor” refers to molecules that precede or follow the biomarker. Thus, once a biomarker is identified as a member of one or more biological pathways, the present invention can include additional members of the biological pathway that come before (are upstream of or a precursor of) or follow (are downstream of) the biomarker. Such identification of biological pathways and their members is within the skill of one in the art.

Cellular markers may be isolated by any suitable method known in the art. Native polypeptide and metabolite markers can be purified from natural sources by standard methods known in the art such as chromatography, centrifugation, differential solubility or immunoassay. In one embodiment, polypeptide and metabolite markers may be isolated from a serum sample using, for example, the chromatographic methods disclosed herein or affinity purification using substrate-bound antibodies that specifically bind to the marker. Metabolite markers may be synthesized using the techniques of organic and inorganic chemistry. Given the amino acid sequence or the corresponding DNA, cDNA, or mRNA that encodes them, polypeptides markers may be synthesized using recombinant or chemical methods. For example, polypeptide markers can be produced by transforming a host cell with a nucleotide sequence encoding the polypeptide marker and cultured under conditions suitable for expression and recovery of the encoded protein from the cell culture.

The present invention also includes the use as biomarkers of polynucleotides that encode any of the polypeptides identified by the methods of the invention or that encode any other polypeptide that can be identified as differentially expressed in subjects with MS using the identification methods of the invention, or that encode a molecule that comprises such a polypeptide or a polypeptide having substantial homology with a component set forth herein.

In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the polynucleotide has been purified, but indicates that the molecule does not include an entire genome or an entire chromosome in which the nucleic acid molecule is found in nature. Polynucleotides useful in the present invention include a portion of a gene (sense or non-sense strand) that is suitable for use as a hybridization probe or PCR primer for the identification of a full-length gene (or portion thereof) in a given sample (e.g., a CSF or serum sample), a gene, or any portion of a gene, as well as a reporter gene.

The minimum size of a polynucleotide of the present invention is a size sufficient to encode a polypeptide having a desired biological activity, sufficient to form a probe or oligonucleotide primer that is capable of forming a stable hybrid with the complementary sequence of a polynucleotide encoding the natural polypeptide, or to otherwise be used as a target in an assay, in a diagnostic assay, or in any therapeutic method discussed herein. The minimum size of a polynucleotide that is used as an oligonucleotide probe or primer is at least about 5 nucleotides in length, and preferably ranges from about 5 to about 50 or about 500 nucleotides or greater (1000, 2000, etc.), including any length in between, in whole number increments (i.e., 5, 6, 7, 8, 9, 10, . . . 33, 34, . . . 256, 257, . . . 500 . . . 1000 . . . ). “Hybridization” has the meaning that is well known in the art, that is, the formation of a duplex structure by two single-stranded nucleic acids due to complementary base pairing.

In one embodiment of the present invention, any amino acid sequence described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

One embodiment of the invention relates to a plurality of polynucleotides for the detection of the expression of biomarkers that are differentially regulated in serum of subjects with MS. The plurality of polynucleotides consists of, or consists essentially of, at least two polynucleotide probes that are complementary to RNA transcripts, or nucleotides derived therefrom, of at least one polynucleotide, the polypeptide encoded by which has been identified herein as being differentially regulated in the serum or CSF of subjects with MS. As such, the plurality of polynucleotides is distinguished from previously known nucleic acid arrays and primer sets. The plurality of polynucleotides within the above-limitation includes at least two or more polynucleotide probes (e.g., at least 2, 3, 4, 5, 6, and so on, in whole integer increments, up to all of the possible probes) that are complementary to RNA transcripts, or nucleotides derived therefrom, of at least one polynucleotide, and preferably, at least 2 or more polynucleotides, encoding polypeptides identified by the present invention. Such polynucleotides are selected from any of the polynucleotides encoding a polypeptide listed in the tables provided herein and can include any number of polynucleotides, in whole integers (e.g., 1, 2, 3, 4, . . . ) up to all of the polynucleotides represented by a biomarker described herein, or that can be identified in MS subjects using the methods described herein. Multiple probes can also be used to detect the same polynucleotide or to detect different splice variants of the same gene. In one aspect, each of the polynucleotides in the plurality is at least 5 nucleotides in length.

The invention also includes antibodies, or antigen binding fragments thereof, that specifically bind to a polypeptide marker, a metabolite marker or a polynucleotide marker, in particular that bind to a component described herein or any other component that can be identified using the methods of the invention. The invention also provides antibodies that specifically bind to a polypeptide having substantial homology with a polypeptide identified herein.

The invention provides antibodies, or antigen binding fragments thereof, that specifically bind to a polypeptide or metabolite of the invention having (i) a mass-to-charge value and (ii) an RT value of about the values stated, respectively, for a marker described herein.

In another embodiment, the invention provides antibodies, or antigen binding fragments thereof, that specifically bind to a component that is a fragment, modification, precursor or successor of a polypeptide or metabolite described herein.

In one embodiment, the present invention provides a plurality of antibodies, or antigen binding fragment, for the detection of biomarkers, the expression of which is differentially regulated in the serum of subjects as described herein. In addition, the plurality of antibodies, or antigen binding fragments thereof, comprises antibodies, or antigen binding fragments thereof, that selectively bind to a biomarker provided herein.

According to the present invention, a plurality of antibodies, or antigen binding fragments thereof, refers to at least 2, at least 3, at least 4, at least 5, at least 6, at least 7, at least 8, at least 9, at least 10, and so on, in increments of one, up to any suitable number of antibodies, or antigen binding fragments thereof, including antibodies representing all of the biomarkers described herein.

According to the present invention, the phrase “selectively binds to” refers to the ability of an antibody or antigen binding fragment thereof to preferentially bind to specified proteins. More specifically, the phrase “selectively binds” refers to the specific binding of one protein to another (e.g., an antibody or antigen binding fragment thereof to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

As used herein, the term “specifically binding,” refers to the interaction between binding pairs such as an antibody and an antigen with an affinity constant of at most 10⁻⁶ moles/liter, at most 10⁻⁷ moles/liter, or at most 10⁻⁸ moles/liter.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Binding partners (e.g., antibodies and antigen binding fragments thereof, or other peptides) useful in any embodiment of the present invention may be conjugated to detectable markers. Detectable labels suitable for use in the present invention include any composition detectable by spectroscopic, photochemical, biochemical, immunochemical, electrical, optical or chemical means, examples of which have been described above. Useful labels in the present invention include biotin for staining with labeled streptavidin conjugate, magnetic beads (e.g., Dynabeads™), fluorescent dyes (e.g., cyanine, tandem, fluorescein, texas red, rhodamine, green fluorescent protein, yellow fluorescent protein and the like), radiolabels (e.g., 3H, ¹²⁵I, ³⁵S, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Specific labels may include AF405, AF647 and/or AF647/750.

The present invention includes the use of any of the biomarkers as described herein (including genes or their RNA or protein products), as targets for the development or identification of therapeutic compositions and strategies for the treatment of MS and/or relapsing MS. More particularly, the present invention includes the use of any of the biomarkers of the invention as targets to identify test compositions that regulate (up or down) the amount, expression or activity of the biomarker or protein or gene or cell represented by any biomarkers described herein.

The present invention also includes a method to diagnose a subject as having multiple sclerosis. The method includes the steps of analyzing a subject sample for the percentage of Th40 cells in a sample isolated from the subject, and diagnosing multiple sclerosis, wherein an increase in the percentage of Th40 cells in the sample from the subject relative to the control sample or standard value indicates multiple sclerosis in the subject.

In an alternative embodiment, the method includes comparison of at least one of the biomarkers in a subject sample with the presence of or amount of one or more of these biomarkers that are present in a sample of a subject known not to have multiple sclerosis. In alternative embodiments, additional indicators and/or biomarkers of multiple sclerosis such as, for example, those indicators and/or biomarkers known in the art to be associated with MS, are used in conjunction with the biomarkers of the present invention to diagnose MS. A preferred sample to test is a serum sample, a whole blood sample or cerebrospinal fluid.

A preferred change, which may indicate that the subject has MS, includes an increase in the percentage of Th40 cells in the sample from the subject, relative to a control sample or standard value of about 50%, about 60%, about 70%, about 75%, about 80%, about 85%, about 90%, and more preferably about 95%.

The present invention also includes a method to diagnose relapse or disease progression in a subject having multiple sclerosis that is being treated for multiple sclerosis. This method includes determining the percentage of Th40 cells in a sample isolated from the subject, comparing the percentage of Th40 cells to a control sample or a standard value and, diagnosing relapse or disease progression, wherein the percentage of Th40 cells in the sample from the subject indicates the subject is in relapse or has disease progression.

A change, which may indicate that the subject has relapse or disease progression of MS, includes an increase in the percentage of Th40 cells in the sample from the subject, relative to a control sample or standard value of about 50%, about 60%, about 70%, about 75%, about 80%, about 85%, about 90%, and more preferably about 95%.

In one embodiment, the present invention includes a method to identify a composition useful for the treatment of multiple sclerosis in a subject. The method includes the steps of administering a test composition to the subject, and evaluating whether administration of the test composition causes a transient change in a biomarker. A biomarker can include CD4, CD40, CD8, CD25, CD45, TCRV8.3+, CXCR3, and CCR5. In this method, a transient change in the biomarker indicates the test composition is useful for treatment of multiple sclerosis. Preferably, such test compositions can be used to further study mechanisms associated with MS or more preferably, serve as a therapeutic agent for use in the treatment or prevention of at least one symptom or aspect of MS, or as a lead composition for the development of such a therapeutic agent.

Using the referenced biomarkers, the method can be used for screening and selecting a test composition, e.g., a chemical compound or a biological compound having regulatory activity as a candidate reagent or therapeutic based on the ability of the composition to affect the expression or activity of the biomarker. Compositions identified in this manner can then be re-tested, if desired, in other assays (e.g., for usefulness as therapeutic compounds) to confirm their activities with regard to the target biomarker or a cellular or other activity related thereto.

As used herein, detecting a biomarker of the present invention can also include detecting transcription of the gene encoding a biomarker protein and/or to detecting translation of the biomarker protein. To detect a biomarker refers to the act of actively determining the level of and/or expression of a biomarker. This can include determining whether the biomarker expression is upregulated as compared to a control, downregulated as compared to a control, or substantially unchanged as compared to a control. Therefore, the step of detecting expression does not require that expression of the biomarker actually is upregulated or downregulated, but rather, can also include detecting no expression of the biomarker or detecting that the expression of the biomarker has not changed or is not different (i.e., detecting no significant expression of the biomarker or no significant change in expression of the biomarker as compared to a control).

Test compositions to be screened in the methods of the invention preferably include homologues and variants of interferons as discussed above. A preferred homologue and/or variant is a homologue or variant of interferon beta.

The present embodiment includes administering the test composition to a subject. A subject is preferably a human. Routes of administration are known in the art, and include intravenous, intraperitoneal, subcutaneous, intramuscular, intragastric, intranasal, intratracheal, inhalational, intracerebral, and dermal routes of administration. A preferred route of administration is intramuscular. Amounts of the composition or test composition to administer can be determined by one of skill in the art, and include clinically acceptable amounts to administer based on the specific activity of the composition and its pharmacodynamic profile.

A preferred biomarker of the present invention includes a monocyte-associated variable. A monocyte-associated variable can refer to a monocyte count, a monocyte/leukocyte ratio, an individual antigen occurring on monocytes, or a soluble factor such as cytokines or metabolites which are associated with monocytes and/or biologically related to monocytes. Generally, for monocyte-associated variable which is an individual antigen or a soluble factor, such as cytokines or metabolites which are associated with monocytes, the most useful variables are ones whose changes are not merely directly proportional to the number of monocytes, rather, the changes are larger than what could be attributed to the change in the monocyte parent population, i.e., are upregulated or downregulated.

Monocytes may be quantitated by any methods known in the art. In a preferred method, monocytes may be quantitated by absolute count (cells/μl blood). Other appropriate methods to quantitate monocytes or any other cell population is to measure a variable that tracks with the cell population, e.g., expression and/or intensity of individual antigens or soluble factors identified by immunoassay and/or mass spectrometry that are biologically related to and therefore proportional to the cell population. In preferred embodiments, the magnitude of the change as measured by any method is at least about 10%, at least about 15%, at least about 20%, at least about 23%, at least about 25%, at least about 30%, at least about 35%, and at least about 38%. In other preferred embodiments, the magnitude of the change as measured by any method has an effect size (difference of means divided by the weighted standard deviation) of at least about 0.2, at least about 0.4, at least about 0.6, at least about 0.8, at least about 1, and at least about 1.2.

Another monocyte-associated variable includes a ratio of monocytes to total number of white blood cells counted, or any subset of white blood cells. In a preferred embodiment, a preferred ratio is the monocyte/leukocyte ratio. Cell populations, e.g., monocytes, leukocytes, and white blood cells can all be quantitated by methods known in the art and as discussed above. In preferred embodiments, the magnitude of the change in the absolute monocyte counts is an increase and is at least about 20%, at least about 30%, at least about 40%, at least about 50%, at least about 55%, at least about 60%, at least about 65%, and even more preferably at least about 70% and at least about 75%, and most preferably between about 50% and about 100%. In other embodiments, the magnitude of change in the monocyte/leukocyte ratio has an effect size of at least about 0.7, at least about 1, at least about 1.2, at least about 1.4, at least about 1.5, and more preferably at least about 1.7, at least about 2, at least about 2.2, and at least about 2.5.

Another monocyte-associated variable includes monocyte expression of HLA Class II molecules. In preferred embodiments, the magnitude of the change is an increase and is at least about 10%, at least about 20%, at least about 30%, at least about 40%, at least about 50%, at least about 55%, at least about 60%, at least about 65%, and even more preferably at least about 70% and at least about 75%, and most preferably between about 50% and about 75%. A preferred change is an increase, which is at least about 30% and preferably between about 30% and about 120%. The values for the change in any biomarker of the present invention may be combined or related with values for any other biomarker and/or otherwise mathematically manipulated for use in methods of the present invention.

In other embodiments, the methods of the present invention include evaluating test compositions, or evaluating a dose of a composition by using in vitro assays which show responses substantially similar to, or equivalent to, the ones discussed herein for human subjects. Any in vitro assay known in the art useful for measurement of the biomarkers discussed herein are included in the embodiments of the present invention. In the case of a cell-based assay, the conditions include an effective medium in which the cell can be cultured or in which the cell lysate can be evaluated in the presence and absence of a composition. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a composition under conditions which take into account the number of cells per container contacted, the concentration of composition administered to a cell, the incubation time of the composition with the cell, and the concentration of composition administered to a cell. Determination of effective protocols can be accomplished by those skilled in the art based on variables such as the size of the container, the volume of liquid in the container, conditions known to be suitable for the culture of the particular cell type used in the assay, and the chemical composition of the composition (i.e., size, charge etc.) being tested. A preferred amount of putative regulatory compound(s) can comprise between about 1 nM to about 10 mM of putative regulatory compound(s) per well of a 96-well plate.

Expression of genes/transcripts and/or proteins encoded by the genes represented by biomarkers of the invention is measured by any of a variety of known methods in the art. In general, expression of a nucleic acid molecule (e.g., DNA or RNA) can be detected by any suitable method or technique of measuring or detecting gene or polynucleotide sequence or expression. Such methods include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of mRNA expressed from one or more of the genes represented by biomarkers of this invention using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

Methods to measure biomarkers of this invention, include, but are not limited to: flow cytometry, laser scanning cytometry, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), hematology analyzer and assays based on a property of the protein including but not limited to DNA binding, ligand binding, or interaction with other protein partners.

In a preferred embodiment, test compositions can be further tested in biological assays that test for other desirable characteristics and activities, such as utility as a reagent for the study of MS or utility as a therapeutic compound for the prevention or treatment of MS. If a suitable therapeutic composition is identified using the methods and genes of the present invention, a composition can be formulated. A composition, and particularly a therapeutic composition, of the present invention generally includes the therapeutic composition and a carrier, and preferably, a pharmaceutically-acceptable carrier.

In another embodiment, the present invention includes a method to identify a dose of a composition, useful for the treatment of multiple sclerosis in a subject, which elicits a desired magnitude of response. This method includes the steps of administering a composition to subjects at different doses, where the composition causes a transient change in the percentage of Th40 cells in a sample isolated from the subject, wherein a transient decrease in the percentage of Th40 cells in samples from the subject indicates the test composition is useful for treatment of multiple sclerosis, and identifying a dose of the composition that elicits the desired magnitude of response in at least one of the biomarkers.

A preferred desired magnitude of response (decrease in the percentage of Th40 cells in a sample isolated from the subject) would be a magnitude of response that is substantially similar to a magnitude of response that is associated with subjects experiencing efficacious treatment of multiple sclerosis and/or relapsing forms of multiple sclerosis. A desired magnitude of response preferably includes a response where at least one biomarker is within about 50%, within about 60%, within about 70%, within about 75%, within about 80%, within about 85%, within about 90%, and more preferably within about 95% of a magnitude of response identified herein. In alternative embodiments, the magnitude of response is evaluated for at least two biomarkers, at least three biomarkers, at least four biomarkers, and at least five or more biomarkers. Preferred magnitude of responses for any of the biomarkers according to this embodiment include effect sizes of expression of any these biomarkers of preferably at least about 0.1, at least about 0.2, at least about 0.3, at least about 0.4, at least about 0.4, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, and at least about 2.8. In preferred embodiments, the magnitude of the change is a decrease in Th40 cell percentage in the subject's blood sample and is at least about 10%, at least about 20%, at least about 30%, at least about 40%, at least about 50%, at least about 55%, at least about 60%, at least about 65%, and even more preferably at least about 70% and at least about 75%, and most preferably between about 50% and about 75%.

In a further embodiment, the present invention includes a method to monitor treatment of a subject for multiple sclerosis. This method includes the steps of administering a therapeutic composition to the subject, and evaluating whether administration of the therapeutic composition causes a transient change in a biomarker. Alternatively, this method can include evaluating whether the magnitude of a transient change decreases over time. In this embodiment, a decrease in the magnitude of a transient change can be predictive of a relapse. Such a change can be caused, for example, by the development of neutralizing antibodies to the therapeutic composition.

A preferred transient change is a transient change that is substantially similar to a transient change that is observed for any of the therapeutically-acceptable forms of interferon beta at dosages which are clinically appropriate. A preferred transient change may be a transient change or transient changes that have been identified. Additionally, a preferred transient change may be chosen by de novo quantitation of the magnitude of response of the herein-described biomarkers for a particular composition.

The present invention also provides assay kits that are suitable for the performance of any method described herein and/or the detection of any of the biomarkers that are described herein. The assay kit preferably contains at least one reagent that is suitable for detecting the expression or activity of a biomarker of the present invention in a test sample (e.g., serum or whole blood), and preferably includes at least one of a probe, PCR primers, an antibody or antigen binding fragment thereof, peptides, binding partners, aptamers, enzymes, enzyme substrates and small molecules that bind to or otherwise identify a biomarker of the invention. The kit can include any reagent needed to perform a diagnostic method envisioned herein or to perform a target-based assay envisioned herein. In one embodiment, the kit can contain a means for detecting a control marker characteristic of a cell type in the test sample. The kit can also include suitable reagents for the detection of and/or for the labeling of positive or negative controls, wash solutions, dilution buffers and the like. The kit can also include a set of written instructions for using the kit and interpreting the results.

The means for detecting of the assay kit of the present invention can be conjugated to a detectable tag or detectable label. Such a tag can be any suitable tag which allows for detection of the reagents used to detect the gene, protein or cells of interest and includes, but is not limited to, any composition or label detectable by spectroscopic, photochemical, electrical, optical or chemical means.

In addition, the means for detecting of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide).

Each reference or publication cited herein is incorporated herein by reference in its entirety.

The following examples are provided for the purpose of illustration and are not intended to limit the scope of the present invention.

### Example S

This study addresses the impact of Th40 cells, a pathogenic effector subset of helper T cells, in MS. MS patients including relapsing/remitting MS (RRMS), secondary progressive MS (SPMS) and primary progressive MS (PPMS) were examined for Th40 cell levels in peripheral blood and the levels were significantly (p<0.0001) elevated compared to controls including healthy non-autoimmune subjects, another non-autoimmune chronic disease, and autoimmune type 1 diabetes. Surprisingly, classically identified Tregs were elevated above controls but the Th40/Treg ratio still predicted autoimmunity. The cohort displayed a wide range of HLA haplotypes including the GWAS identified predictive HLA-DRB1*1501 (DR2). However half the subjects did not carry DR2 and regardless of HLA haplotype, Th40 cells were expanded during disease. In RRMS Th40 cells demonstrated a limited TCR clonality. Mechanistically, Th40 cells demonstrated a wide array of response to CNS associated self-antigens that was dependent upon HLA haplotype. Th40 cells were predominantly memory phenotype producing IL-17 and IFNγ with a significant portion producing both inflammatory cytokines simultaneously suggesting an intermediary between Th1 and Th17 phenotypes.

### Introduction:

The studies disclosed in this Example demonstrate that MS subjects have a significantly expanded number of Th40 cells (CD4⁺CD40⁺) compared to control subjects or T1D patients in peripheral blood. In a cohort of 48 patients, HLA haplotypes were determined and as expected HLA-DR15 and DQ6 were predominant, but DR3, DR4, and DQ8 subjects that are more closely associated with T1D and rheumatoid arthritis rather than MS were identified. Regardless of HLA expression Th40 cell levels were significantly elevated during MS, suggesting a measure other than HLA that correlates more consistently with disease occurrence. Th40 cells from MS patients typically demonstrated a clonal expansion of TCRVα8.3⁺ cells and recognized CNS antigens including MBP, MOG and PLP peptides in an HLA haplotype restricted manner. Th40 cells in MS are predominantly memory phenotype and co-express CXCR3 and CCR5. Th40 cells predominantly produce IL-17, but a significant portion produce both IL-17 and IFNγ simultaneously. These data suggest a possible biomarker that not only associates a definable T cell subset with MS, but improves evidence for a new autoimmune based, rapid blood test that avoids the more invasive spinal tap, for MS disease diagnosis.

### Materials and Methods:

Patients:

Subjects diagnosed with multiple sclerosis, meeting the McDonald criteria, were recruited from the Rocky Mountain Multiple Sclerosis Center and the Department of Neurology of the University of Colorado Denver Medical Campus. Patients included relapsing/remitting (RRMS), secondary progressive (SPMS), primary progressive and control subjects. Autoimmune controls included type 1 diabetes (T1D) subjects recruited from the Barbara Davis Childhood Diabetes Center (BDC), Denver Colo. T1D subjects meet American Diabetes Association and the National Institutes of Health criteria for disease. Chronic disease controls included diagnosed T2D patients with no reported infections. Subjects had no infections or complicating diseases at time of examination. All subjects were recruited under a Colorado Multiple Institutional Review Board (COMIRB) approved protocol and signed informed consent forms. Our human participant policy conforms to the uniform requirements of the International Committee of Medical Journal Editors.

| | Controls | T1D | T2D | CDMS |
|---|---|---|---|---|
| Age Range | 17-63 | 12-65 | 22-62 | 20-58 |
| Median Age | 40.2 ± 11.4 | 40.3 ± 8.5, | 47.73 ± 10.5 | 41.73 ± 10.5 |
| Average Disease | N/A | 15 years | 17 years | 12 years |
| Duration | | | | |
| Gender: | 42/40 | 48/47 | 32/36 | 42/26 |
| Female/Male | | | | |
| (Number of | | | | |
| subjects) | | | | |

Blood Samples:

Venal blood was drawn into tubes with an anti-coagulant, diluted 1:1 with PBS and separated over a ficoll gradient that was centrifuged for 30 minutes at 400×g. The layer of PBMC was removed from the buffy coat and rinsed twice; first in running buffer (PBS, 0.5% BSA, 0.2 mM EDTA) and then in Aim-V medium (Invitrogen, Grand Island, N.Y.) at slow speed (200×g) to remove residual platelets.

Staining:

Cells were stained using antibodies for CD4, CD8, CD25, and CD45 that were purchased from Miltenyi (Auburn, Calif.) and FoxP3, IFNγ, and IL-17A were purchased from eBioscience (San Diego, Calif.). Anti-CD40 (clone G28-5) maintained in house conjugated to AF405, AF647 or AF647/750. Cells were incubated with appropriate antibodies for 30 min, and washed with running buffer, PBS, 0.5% BSA, 0.2 mM EDTA. Intracellular cytokine staining was performed by treating with eBioscience permeabilization/fixation buffer for 10 min, and then incubated with antibodies for 10 min. Cells were washed and assayed on a Miltenyi Macs-Quant bench-top flow cytometer with 7 color compensations determined for human cells.

Cell Purification:

Following Ficoll purification of PBMC, cells were incubated with anti-HLA antibody micro-beads (Miltenyi) for 30 minutes with gentle rocking Cells were passed through a Miltenyi AutoMACS set on deplete-s. Positive fraction was considered antigen presenting cells (APC). Helper T cells were purified by sorting HLA-depleted fractions with CD4 micro-beads (Miltenyi) followed by AutoMACS set to possel-s. Cell purity was >98% as determined by staining and flow cytometry.

Antigen Assays:

Purified APC were exposed to antigens at concentration of 5 μg/ml in a 96 well u-bottom plate for 8-12 hours and then the cells were twice exposed to a UV light source UV Strata-link 2400 (Agilent Technologies, Santa Clara Calif.) for 1 minute to irradiate APC. MS associated antigens include:

MOG₃₅₋₅₅, MEVGWYRPPFSRVVHLYRNGK;

MOG₉₉₋₁₀₇, FFRDHSYQE;

PLP₁₀₃₋₁₂₀, LVERGNHSKIVAGVLGLI;

MBP₁₄₆₋₁₇₀ AQGTLSKIFKLGGRDSRSGSPMARR; and

MBP₈₃₋₁₀₂, ADPGSRPHLIRLFSRDAPGR.

PEDIACEL, composed of purified diphtheria toxoid, tetanus toxoid, acellular pertussis vaccine, inactivated poliovirus, and haemophilus influenza type b polysaccharide was used as a positive control. CD4 cells were labeled with CFSE and 5×10⁴ cells were incubated in 96 well plates with equivalent numbers of APC. Cells were incubated for 7 days then stained with CD4, CD8, CD40, CD25 and CD45 and examined via flow-cytometry using the Miltenyi Macs-Quant.

HLA Testing:

HLA haplotypes were determined by the HLA core facility at the Barbara Davis Childhood Diabetes Center at the University of Colorado Denver Anschutz Medical Campus. HLA is determined by RT-PCR using appropriate primers for HLA-DRs and DQs from blood cells.

### Results:

Th40 T Cells are Prevalent in MS:

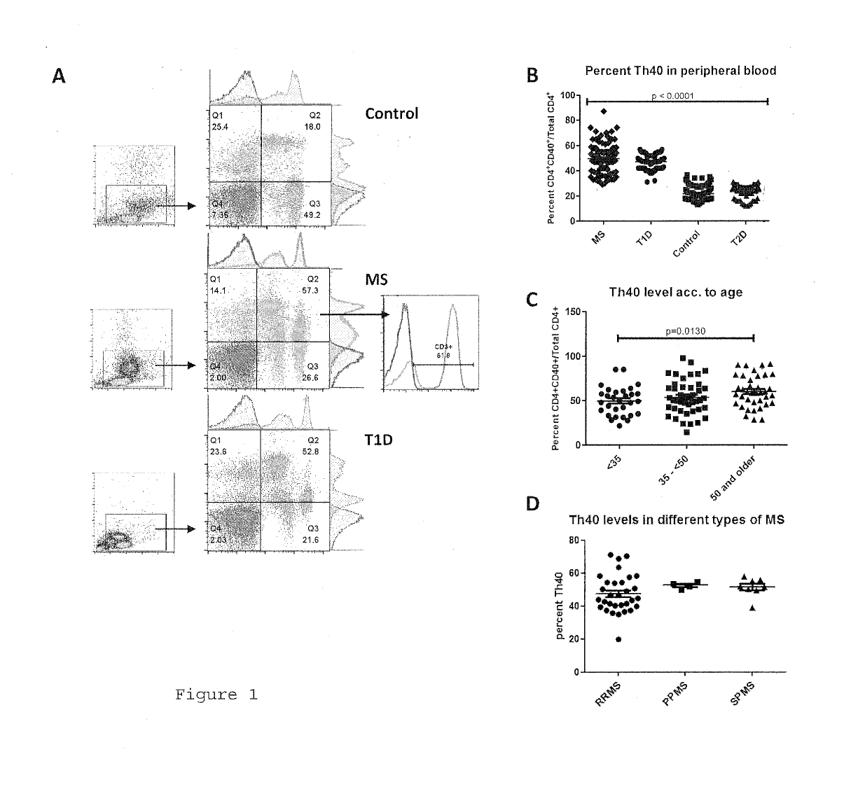

In a blinded study, we examined peripheral blood to determine levels of Th40 cells. Subjects diagnosed with MS were compared to non-autoimmune controls and to an established autoimmune disease, type 1 diabetes as well as to a chronic but non-autoimmune disease. MS subjects (41.73±10.5 years old) were age matched to non-autoimmune (40.2±11.4 years old) and to T1D (40.3±8.5,) autoimmune control subjects. Like in T1D, MS subjects have a substantially increased percentage of Th40 cells in peripheral blood when compared to non-autoimmune controls (FIG. 1 A). However MS subjects demonstrate a significantly (p<0.01) elevated percentage of Th40 cells when compared to T1D patients. Th40 cells from MS subjects express high levels of CD3, a classic T cell molecule (FIG. 1A). In MS subjects approximately half of the CD40⁺ T cells are CD4^{hi} with the remainder being CD4^{lo}. The majority of CD40 expressing cells from T1D patients occur as CD4^{lo} (FIG. 1A). In previous work we showed by western blot that while the cell surface expression of CD4 is lower, the overall cellular CD4 levels are identical (Vaitaitis and Wagner, 2008). This suggests cell surface down-regulation of CD4, potentially constituting an activated or memory phenotype status. Because of the CD4^{lo} status, this population of cells typically has been overlooked.

MS is a chronic disease; therefore we compared Th40 levels from type 2 diabetes (T2D) patients, a chronic but non-autoimmune disease (FIG. 1B). Th40 cell percentages from T2D subjects were identical to non-autoimmune controls; thus the chronic disease state does not account for the expansion of Th40 cells (FIG. 1B). The Th40 percentages demonstrate a broader range in MS (33% to 87%) compared to T1D or controls (FIG. 1B). We speculated that the relapsing/remitting course may be reflected in Th40 cell levels so we further characterized levels of Th40 cells comparing RRMS versus PPMS and SPMS (FIG. 1C). The cohort of PPMS and SPMS was smaller than RRMS; but levels of Th40 were more tightly grouped in PPMS and SPMS. The wide range of Th40 cells occurred in RRMS, which may reflect the variable nature of the relapsing/remitting disease course.

Tregs in MS:

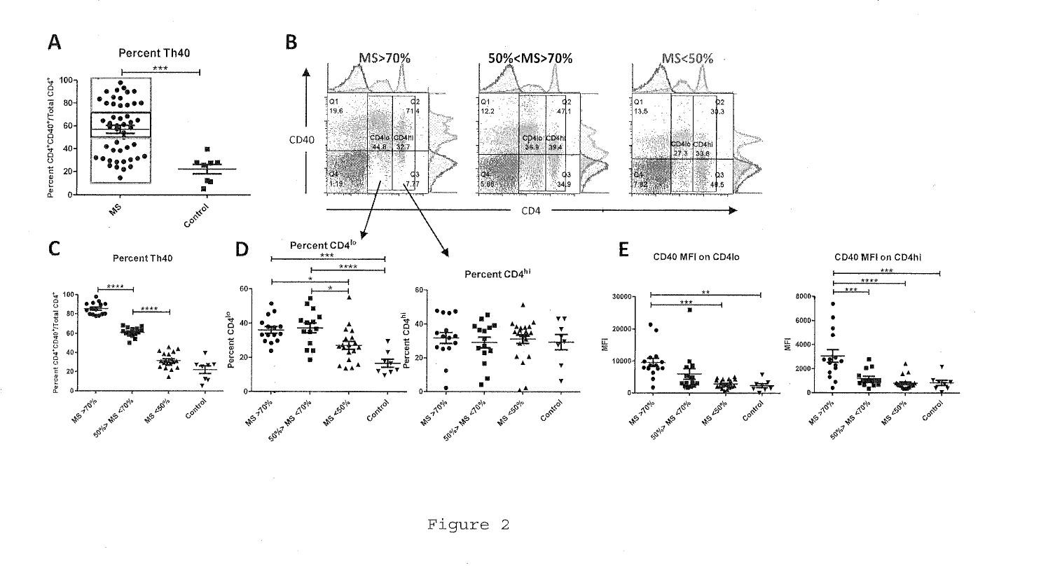

A seminal question in autoimmunity is the role of Tregs. It is assumed that during autoimmunity, Tregs, which are responsible for some degree of tolerance, are somehow deficient, either in total number or in function. Furthermore that deficiency is presumed to be a predominant source for breach of tolerance. In the mouse model of T1D we recently reported that pathogenic effector cells can express then lose Foxp3 (Vaitaitis et al., 2013). It is important therefore to correctly define Tregs. We compared the percentage of classically defined Tregs, CD4⁺CD25⁺FOXP3⁺ cells, in MS subjects to Tregs from non-autoimmune control and T1D patients (FIG. 2A). A surprising result was that Tregs were slightly but significantly elevated (p=0.0369) in peripheral blood of our MS cohort compared to controls (FIG. 2A). This finding is counter-intuitive given the potential role for Treg mediated tolerance. We further compared Treg percentages from age matched T1D subjects, representing a chronic autoimmune disease. Tregs from T1D subjects were significantly lower than levels in MS subjects but also were significantly (p<0.0001) lower than non-autoimmune controls (FIG. 2A). This observation would be expected if Treg numbers or function is dysregulated. The MS cohort in this study was all RRMS. Even though Treg percentages in MS subjects are elevated relative to controls, the ratio of Th40 to Treg (4.2±0.24) in MS is significantly elevated compared to non-autoimmune controls (1.7±0.86) [FIG. 2B]. The difference in Th40 to Treg ratio is further exaggerated in T1D (FIG. 2B). These data may suggest that dysregulation lies more prominently in Th40 cells rather than Tregs in MS, but in T1D dysregulation may occur in both Tregs and Th40 cells.

Th40 Levels Relative to HLA Haplotype:

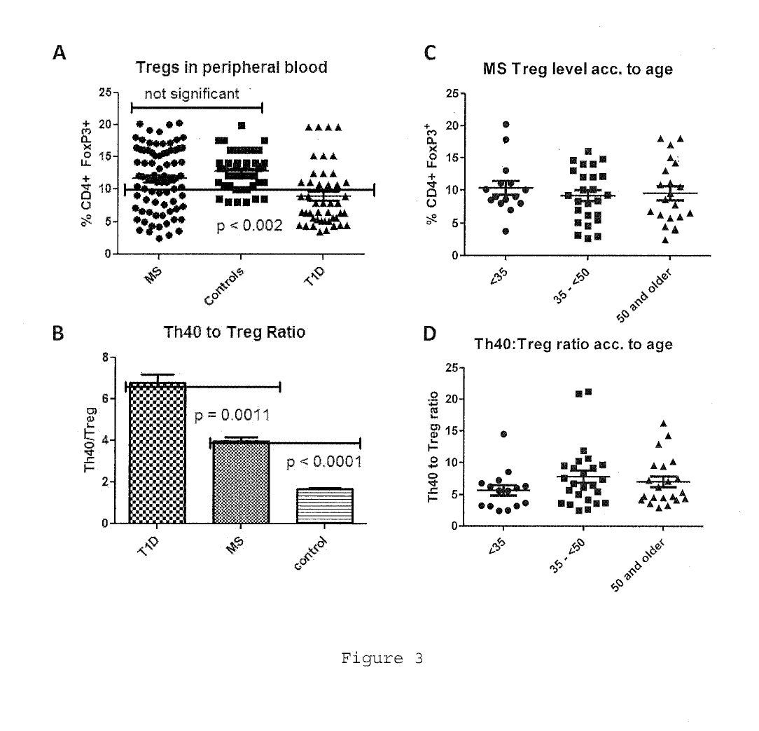

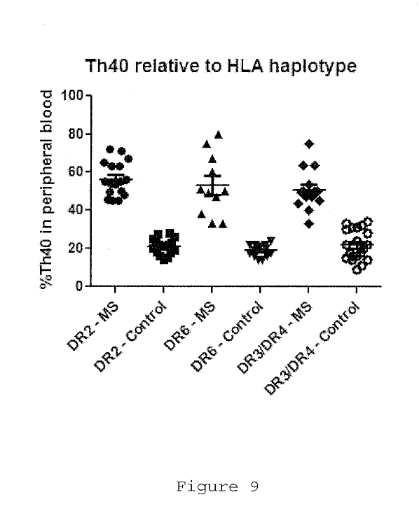

Genome wide association studies (GWAS) have provided some clues for biomarkers in autoimmune diseases including MS. For example, the most reliable and descriptive biomarker in MS thus far determined is HLA-DRB1*1501 originally known as HLA-DR2 but also referred to as DR15 (Hoffjan and Akkad, 2010; International Multiple Sclerosis Genetics et al., 2007; Sawcer et al.). We examined HLA expression in a cohort of 48 MS subjects that included RRMS and SPMS patients. As predicted, the most common HLA-DR allele expressed was HLA-DRB 1*1501 (DR2 or DR15). Interesting though, in our cohort HLA-DR6 occurred as frequently as HLA-DR2 (FIG. 3A). The third most commonly occurring allele was HLA-DR4, which is associated with type 1 diabetes and rheumatoid arthritis (Angelini et al., 1992; Thorsby and Ronningen, 1993). We found that many MS subjects from our cohort did not carry the DR2 haplotype. In fact several MS subjects carried DR4/DR3 that is highly associated with T1D (Steck and Rewers, 2011) (FIG. 3A). In our MS cohort 2 subjects demonstrated T1D comorbidity. Th40 percentages were determined relative to each HLA-DR haplotype. Although a wide range of Th40 percentages occurred, the mean percentage of Th40 cells did not statistically alter relative to any HLA haplotype (FIG. 3 B). The widest range of Th40 cell levels in MS occurred within the DR6 and DR2 haplotypes (range=64). Subjects carrying DR1 and DR3 had the narrowest range of Th40 cell percentages (range=17 and 18.5 respectively). In subjects whose Th40 cell percentages were above 60% the more common HLA haplotypes were DR6 and DR2, although several subjects carrying DR3 or DR4 had Th40 cell percentage above 60%. Importantly for each HLA haplotype in MS, the Th40 level was significantly (p<0.001) increased compared to HLA matched controls (FIG. 9). This included MS subjects who do not carry the HLA-DR2, MS predictive haplotype.

We examined the DQ alleles finding a predominance of DQ6 (FIG. 3C), which was expected. In our cohort DQ7, DQ2 and DQ5 were sequentially the next most common haplotypes (FIG. 3C). For Th40 levels, the same observations occurred for HLA-DQ haplotypes as for DR haplotypes; there were no statistical differences in Th40 cell levels relative to DQ allele expression (FIG. 3D). These data demonstrate that regardless of HLA haplotype during MS, Th40 cell levels are significantly increased relative to controls. Importantly in MS patients who do not carry the predictive HLA-DRB1*1501 allele, those subjects still had elevated Th40 cell levels (FIG. 3B and FIG. 9). Conversely, several non-autoimmune control subjects do carry the HLA-DRB1*1501 allele, but do not have elevated Th40 cell levels (FIG. 9). These data argue that Th40 cell percentages are independent of HLA haplotype and are a stronger predictor for MS susceptibility or diagnosis.

T Cell Limited Clonality in MS:

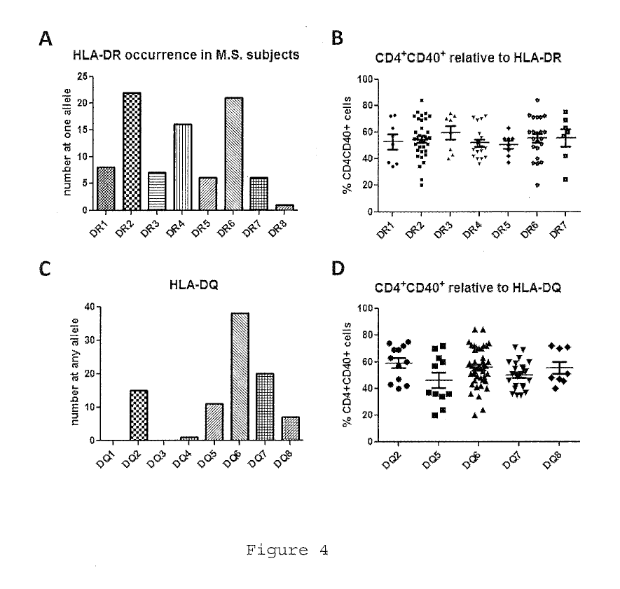

Models for autoimmune disease often demonstrate T cell clonal expansions (Fox, 1996; Wucherpfennig and Hafler, 1995). In the EAE model of MS, T cell clonality has been suggested (Harrington et al., 1998; Huseby et al., 2001). TCR transgenic mice have been generated that spontaneously develop EAE (Goverman et al., 1993) further emphasizing clonal contributions in the disease process. We examined for a clonal expansion in our MS cohort, focusing on Th40 cells from RRMS patients. In a cohort of 48 patients, we determined that 37.5% demonstrated a detectable increase (16.2%±4.3%) in the TRAV8-3 gene product TCR Vα8.3 (FIG. 4). This particular TCR Vα has been associated with autoimmunity in human subjects (Kent et al., 2005). TCR Vα usage was not examined; therefore true clonality was not assessed. The majority of those TCR Vα8.3 cells (83.4%±6.4%) were CD45RO⁺, a memory phenotype (FIG. 4). In none of the subjects examined did the CD4⁺CD40⁻ cells demonstrate TCR Vα8.3 expansions (FIG. 4).

Th40 Cell Response to CNS Antigens is HLA Restricted:

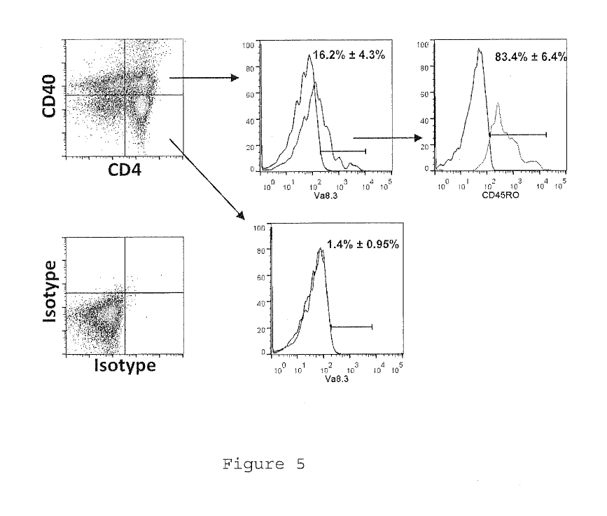

As a disease predictive mechanistic approach we addressed whether Th40 cells from MS subjects are Central Nervous System (CNS) self-antigen responsive. The antigens were peptide derivatives from MOG, MBP and PLP proteins each of which has been associated with MS and the EAE model of the disease (Arbour et al., 2006). Analysis was done relative to HLA haplotype. When the DR2 allele was present at one allele with the other allele being any haplotype, the best antigen response by Th40 cells was to MOG₉₉ peptide followed by the PLP₁₀₃ peptide (FIG. 5A). The least reactive peptide was MBP₈₃. It is notable however that there was no statistical difference between any of the peptides that were tested. When the DR6 haplotype was a fixed allele and any haplotype occurred at the other allele, Th40 cells responded quite differently to the antigens (FIG. 5B). The peptides inducing the best response were MBP₈₃ and PLP₁₀₃.). In our cohort there were no DR2/DR2 individuals. When DR6 occurred at both alleles, the PLP₁₀₃ antigen induced the best Th40 response, which was significantly above MBP₁₄₆ (the worst response antigen) or either MOG peptide (FIG. 5C). The most variation of response (most significant differences) occurred when DR2 and DR6 were co-expressed the prominent response antigen was MOG₃₅ (significantly greater than any of the tested antigens including the PED positive control) followed by PLP₁₀₃ that was significantly greater than the remaining antigens tested (FIG. 5D). There was no response to MBP₁₄₆ and significantly less response to MOG₉₉.

The third most common DR allele expressed in our MS cohort was DR4, which GWAS has associated with T1D susceptibility (Steck and Rewers, 2011). Interestingly Th40 cells from DR4 expressing MS subjects responded to MOG₉₉ and equally to PLP₁₀₃ and respond to both MBP peptides with very little response to MOG₃₅ (FIG. 5E). When DR4 is co expressed with DR2 the response to MOG₉₉ remains high while response to MBP₈₃ is diminished (FIG. 5F). When DR4 is co expressed with DR6 the MOG99 response is unaffected, but response to MBP83 is increased (FIG. 5G). Cumulatively these data demonstrate that Th40 cells do respond to classic MS associated antigens, but with varying degrees depending upon the HLA haplotypes can present the described antigens. The data suggest a varied TCR repertoire within Th40 cells, but also suggest epitope spreading; this is because the total response across all antigens is greater than 100%. Epitope spreading has been well described in EAE the mouse model for MS (McMahon et al., 2005; Miller et al., 2001; Miller et al., 2007; Smith and Miller, 2006; Zhang et al., 2004). These data suggest that some Th40 cells are responding to more than one single peptide.

Th40 T Cells and Memory/Effector Phenotype:

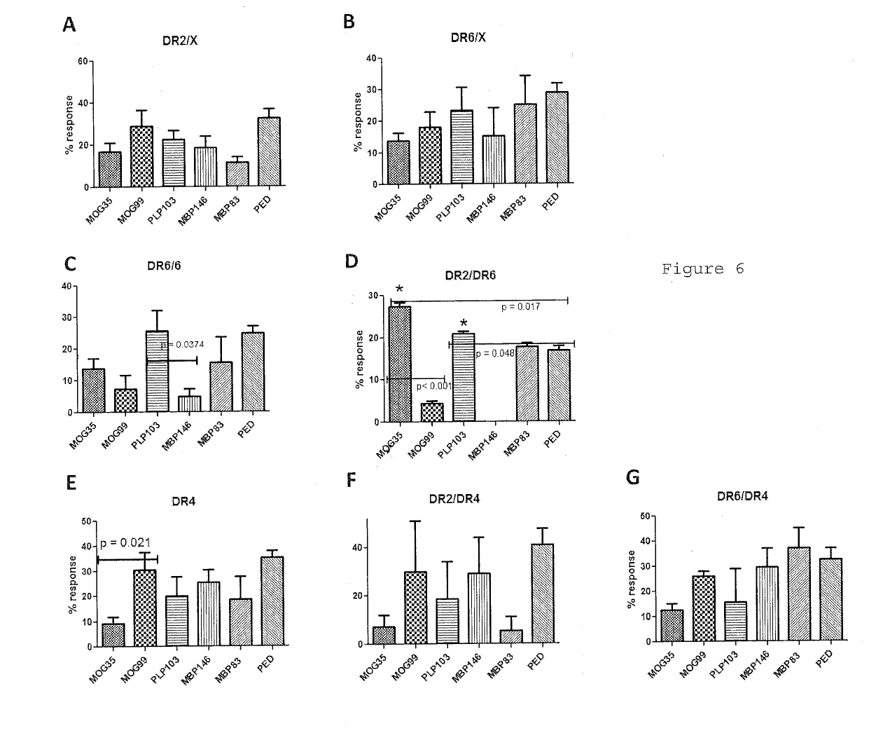

We further characterized memory phenotype within the total Th40 cell population from MS subjects. The majority (67.13%±8.1%) of Th40 cells from MS subjects were CD45RO⁺, a memory T cell biomarker (FIG. 6A); while a significantly (p<0.001) smaller portion (34±12.6%) of the CD4⁺CD40⁻ T cell population was CD45RO⁺. Non-autoimmune subjects had significantly fewer Th40 T cells overall and a smaller portion (36%±6.2) of Th40 cells were CD45RO⁺ (FIG. 6B). Furthermore, there was no difference between Th40 T cells and CD4⁺CD40⁻ T cells with regard to CD45RO expression in those control subjects (FIG. 6B). These data demonstrate that MS results in increases in memory T cells within the Th40 cell population and not within CD4⁺CD40⁻ cells. It has been reported that memory T cells from MS patients do not rely on CD28 signaling (Lovett-Racke et al., 1998). CD40 acts as a T cell co-stimulus (Baker et al., 2008), therefore an important alternative for memory T cell costimulation, CD40, emerges. An additional feature examined was the chemokine receptors CCR5 and CXCR3, which are associated with T cell trafficking We previously reported that Th40 cells in T1D patients predominantly express CXCR3 (Waid et al., 2007). Unlike T1D, a larger number of MS patients had Th40 cells that expressed significantly increased (p<0.001) levels of CCR5 or co-expressed CCR5 and CXCR3 (FIG. 6C).

Th40 Cells and Cytokine Production in MS:

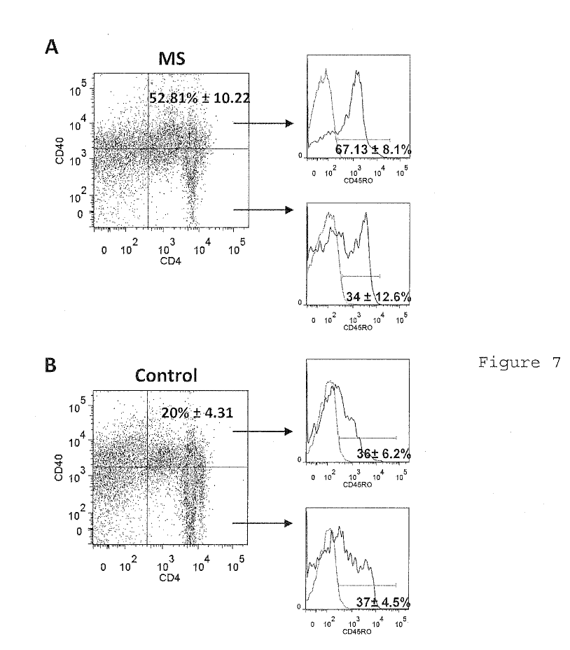

Inflammatory cytokines including IFNγ, a Th1 cytokine (Anderson and Rodriguez) and IL-17A, the defining cytokine for the Th17 subset (Graber et al., 2008) are reported to promote MS progression. We determined that memory Th40 cells from MS patients produce IFNγ and IL-17 (FIGS. 7A and B). Lymphocytes from MS subjects were isolated and were gated on CD4 and CD45RO then further CD40 gated (FIG. 7 A) and cytokine levels were determined by intracellular staining A small portion (8.4%±4) of peripheral memory Th40 cells produced IFNγ alone (FIG. 7 B). A substantial proportion (30%±10.6) produced IFNγ⁺ and IL-17⁺ and an equal proportion (39.4%±10) produced IL-17⁺ alone (FIG. 7). The IFNγ/IL17 double producers and the IL-17 only producers were significantly increased above IFNγ alone (FIG. 7B). Th40 cells from control, non-autoimmune, non-infected subjects did not produce either cytokine immediately ex vivo (data not shown). It should be noted that cells were classic memory phenotype and gated exclusively on the Th40 population. Cells were analyzed by intracellular staining, which means that the cells were primed to produce cytokines, but not necessarily activated to do so.

### Discussion:

Multiple Sclerosis is a complicated, neurodegenerative disease with a yet undetermined etiology. Genome-wide association studies (GWAS) indicate no neural associated genes but do indicate immune related genes (Hafler et al., 2007). Genes identified include HLA-DRB1*1501 (DR15), DR6 and DQ6 each located within major histocompatibility complex (MHC) gene sets, the IL-7 receptor (CD127) and IL-2 receptor genes (Hafler et al., 2007; Sawcer, 2008). IL-7 and IL-2 are critical cytokines in T cell development and interestingly CD127^{lo} cells are associated with regulatory T cell (Treg) designation (Liu et al., 2006). HLA expression impacts T cell development thus GWAS points towards T cell abnormalities during MS. Epidemiological analysis convincingly show that some genetic factors and immune related systems are relevant but lack power to illuminate the extent to which these factors are involved in MS (Sawcer, 2008). Several different autoimmune diseases have been examined using GWAS and compared to each other as well as inflammatory non-chronic infectious disease. Rather than revealing significant differences as was expected, studies reveal significant similarities, the majority of which are immune related specifically involving T cells.

We identified a CD4⁺ T cell subset expressing the CD40 receptor and showed that those T cells directly impact autoimmune pathogenesis (Carter et al., 2012a; Siebert et al., 2008; Vaitaitis and Wagner Jr., 2010; Vaitaitis et al., 2010; Vaitaitis et al., 2003; Vaitaitis and Wagner, 2008; Vaitaitis and Wagner, 2010; Vaitaitis and Wagner, 2012; Wagner et al., 2002; Waid et al., 2008; Waid et al., 2004; Waid et al., 2007). Th40 cells passively transfer type 1 diabetes in mouse models but importantly are involved in human disease (Siebert et al., 2008; Waid et al., 2007). As autoimmunity develops, Th40 cells increase in percentage and increase in number systemically (Vaitaitis et al., 2003; Wagner et al., 2002; Waid et al., 2008; Waid et al., 2004). We determined that Th40 cell levels do not wax and wane throughout autoimmunity as other ‘activated’ cells e.g. HLA⁺, CD30⁺, CD25⁺, CD69⁺ or CD154⁺, do. In longitudinal studies in human T1D, Th40 cell levels are as high in subjects who were diagnosed greater than 45 years previously as to those who were diagnosed 2 weeks prior (Siebert et al., 2008; Waid et al., 2007). In human T1D studies the Th40 occurrence remains relatively close to the disease associated mean, however in MS subjects Th40 cell levels varied a great deal relative to the mean. The majority of subjects examined were RRMS thus the variance in Th40 cell levels may reflect the relapsing/remitting nature of the disease. In fact RRMS subjects demonstrate wide Th40 variation while SPMS and PPMS have less variation.

T cell activation requires two signals: 1) TCR mediated signal that leads to expression of activation molecules including CD69, CD25 and CD154 (Nyakeriga et al., 2012) and co-stimulus (Bretscher, 1992). In the case of autoimmunity TCR recognition of self-antigen would necessarily lead to expression of classic activation markers and once the antigen source is diminished, T cell activation should wane. In MS, CD69⁺ T cells return to levels close to that for non-autoimmune controls (Ichikawa et al., 1996), suggesting that early activation conditions diminish during disease. In this report we showed that a cohort of RRMS patients demonstrates TCR clonality within the Th40 cell populations and Th40 cells are responsive to MS associated antigens. Importantly Th40 cells are responsive to several different CNS antigens, suggesting a broad repertoire array localized within those T cells. Because of this diversity, Th40 cells would be primed to prolong pathogenesis and as shown here, likely through production of the inflammatory cytokines IFNγ and IL-17.

The data revealed that depending on HLA haplotype, individual MS subjects were responsive to different MS associated self-antigens, suggesting multiple pathways to disease onset. Also within any individual the same T cell subset could be responsive to different self-antigens. This poses a unique problem relative to therapeutic design. Copaxone is a hexamer peptide derived from the myelin basic protein (MBP) sequence that is commonly used to treat MS. Attempts for tolerance induction towards a single self-antigen may ultimately prove fruitless; given the overall plasticity of the immune system and now plasticity of individual immune responses in MS. A critical element here is epitope spreading, which has been established in EAE the mouse model of MS (McMahon et al., 2005; Miller et al., 2007; O'Connor et al., 2005; Smith and Miller, 2006). If pathogenic effector cells can respond to a wide range of self-antigens, then pinning down a single antigen would prove very difficult. Alternative approaches to tolerance induction in MS now include multiple antigen treatments. In a human trial study of MS patients, an infusion of autologous blood mononuclear cells that were chemically coupled to 7 different MS associated peptides including MOG₃₅ and MBP₈₃, which were used in the this study, demonstrated a decrease in antigen specific T cell responses (Lutterotti et al., 2013). This is encouraging but autoimmunity will likely require a multi-faceted treatment approach. By identifying a biomarker, in this case CD40, which is associated with long-term pathogenic T cells and specifically respond to MOG₃₅ and MBP₈₃, among other antigens, a new approach to tolerance emerges; inducing anergy directly through CD40 blockade. Previous attempts at universal pathogenic T cell control have focused on controlling CD28 or T cell activation steps mediated by ZAP70. These attempts have not been successful. By considering the actions of CD40, specifically directed towards pathogenic T cells, controlling autoimmune inflammation may be possible.

### REFERENCES

Anderson, G., Rodriguez, M., Multiple sclerosis, seizures, and antiepileptics: role of IL-18, IDO, and melatonin. Eur J Neurol 18, 680-685.
Angelini, G., Morozzi, G., Delfino, L., Pera, C., Falco, M., Marcolongo, R., Giannelli, S., Ratti, G., Ricci, S., Fanetti, G., et al., 1992. Analysis of HLA DP, DQ, and DR alleles in adult Italian rheumatoid arthritis patients. Hum Immunol 34, 135-141.
Arbour, N., Lapointe, R., Saikali, P., McCrea, E., Regen, T., Antel, J. P., 2006. A new clinically relevant approach to expand myelin specific T cells. J Immunol Methods 310, 53-61.
Baker, R. L., Wagner, D. H., Jr., Haskins, K., 2008. CD40 on NOD CD4 T cells contributes to their activation and pathogenicity. J Autoimmun 31, 385-392.
Benveniste, E. N., Nguyen, V. T., Wesemann, D. R., 2004. Molecular regulation of CD40 gene expression in macrophages and microglia. Brain Behav Immun 18, 7-12.
Bretscher, P., 1992. The two-signal model of lymphocyte activation twenty-one years later. Immunology Today 13, 74-76.
Carter, J., Vaitaitis, G. M., Waid, D. M., Wagner, D. H., 2012a. CD40 engagement of CD4(+) CD40(+) T cells in a neo-self antigen disease model ablates CTLA-4 expression and indirectly impacts tolerance. Eur J Immunol 42, 424-435.
Carter, J., Vaitaitis, G. M., Waid, D. M., Wagner, D. H., Jr., 2012b. CD40 engagement of CD4+CD40+ T cells in a neo-self antigen disease model ablates CTLA-4 expression and indirectly impacts tolerance. Eur J Immunol 42, 424-435.
Fisniku, L. K., Brex, P. A., Altmann, D. R., Miszkiel, K. A., Benton, C. E., Lanyon, R., Thompson, A. J., Miller, D. H., 2008. Disability and T2 MRI lesions: a 20-year follow-up of patients with relapse onset of multiple sclerosis. Brain 131, 808-817.
Fox, R. I., 1996. Clinical features, pathogenesis, and treatment of Sjogren's syndrome. Curr Opin Rheumatol 8, 438-445.
Girvin, A. M., Dal Canto, M. C., Miller, S. D., 2002. CD40/CD40L interaction is essential for the induction of EAE in the absence of CD28-mediated co-stimulation. J Autoimmun 18, 83-94.
Giuliani, F., Hader, W., Yong, V. W., 2005. Minocycline attenuates T cell and microglia activity to impair cytokine production in T cell-microglia interaction. J Leukoc Biol 78, 135-143.
Goverman, J., Woods, A., Larson, L., Weiner, L. P., Hood, L., Zaller, D. M., 1993. Transgenic mice that express a myelin basic protein-specific T cell receptor develop spontaneous autoimmunity. Cell 72, 551-560.
Graber, J. J., Allie, S. R., Mullen, K. M., Jones, M. V., Wang, T., Krishnan, C., Kaplin, A. I., Nath, A., Kerr, D. A., Calabresi, P. A., 2008. Interleukin-17 in transverse myelitis and multiple sclerosis. J Neuroimmunol 196, 124-132.
Hafler, D. A., Compston, A., Sawcer, S., Lander, E. S., Daly, M. J., De Jager, P. L., de Bakker, P. I., Gabriel, S. B., Mirel, D. B., Ivinson, A. J., Pericak-Vance, M. A., Gregory, S. G., Rioux, J. D., McCauley, J. L., Haines, J. L., Barcellos, L. F., Cree, B., Oksenberg, J. R., Hauser, S. L., 2007. Risk alleles for multiple sclerosis identified by a genomewide study. N Engl J Med 357, 851-862.
Harrington, C. J., Paez, A., Hunkapiller, T., Mannikko, V., Brabb, T., Ahearn, M., Beeson, C., Goverman, J., 1998. Differential tolerance is induced in T cells recognizing distinct epitopes of myelin basic protein. Immunity 8, 571-580.
Hemmer, B., Archelos, J. J., Hartung, H. P., 2002. New concepts in the immunopathogenesis of multiple sclerosis. Nat Rev Neurosci 3, 291-301.
Hoffjan, S., Akkad, D. A., 2010. The genetics of multiple sclerosis: an update 2010. Mol Cell Probes 24, 237-243.
Huseby, E. S., Liggitt, D., Brabb, T., Schnabel, B., Ohlen, C., Goverman, J., 2001. A pathogenic role for myelin-specific CD8(+) T cells in a model for multiple sclerosis. J Exp Med 194, 669-676.
Ichikawa, H., Ota, K., Iwata, M., 1996. Increased Fas antigen on T cells in multiple sclerosis. J Neuroimmunol 71, 125-129.
International Multiple Sclerosis Genetics, C., Hafler, D. A., Compston, A., Sawcer, S., Lander, E. S., Daly, M. J., De Jager, P. L., de Bakker, P. I., Gabriel, S. B., Mirel, D. B., Ivinson, A. J., Pericak-Vance, M. A., Gregory, S. G., Rioux, J. D., McCauley, J. L., Haines, J. L., Barcellos, L. F., Cree, B., Oksenberg, J. R., Hauser, S. L., 2007. Risk alleles for multiple sclerosis identified by a genomewide study. N Engl J Med 357, 851-862.
Kent, S. C., Chen, Y., Bregoli, L., Clemmings, S. M., Kenyon, N. S., Ricordi, C., Hering, B. J., Hafler, D. A., 2005. Expanded T cells from pancreatic lymph nodes of type 1 diabetic subjects recognize an insulin epitope. Nature 435, 224-228.
Liu, W., Putnam, A. L., Xu-Yu, Z., Szot, G. L., Lee, M. R., Zhu, S., Gottlieb, P. A., Kapranov, P., Gingeras, T. R., Fazekas de St Groth, B., Clayberger, C., Soper, D. M., Ziegler, S. F., Bluestone, J. A., 2006. CD127 expression inversely correlates with FoxP3 and suppressive function of human CD4+ T reg cells. J Exp Med 203, 1701-1711.
Lovett-Racke, A. E., Trotter, J. L., Lauber, J., Perrin, P. J., June, C. H., Racke, M. K., 1998. Decreased dependence of myelin basic protein-reactive T cells on CD28-mediated costimulation in multiple sclerosis patients. A marker of activated/memory T cells. J Clin Invest 101, 725-730.
Lucchinetti, C. F., Popescu, B. F., Bunyan, R. F., Moll, N. M., Roemer, S. F., Lassmann, H., Bruck, W., Parisi, J. E., Scheithauer, B. W., Giannini, C., Weigand, S. D., Mandrekar, J., Ransohoff, R. M., 2011. Inflammatory cortical demyelination in early multiple sclerosis. N Engl J Med 365, 2188-2197.
Lutterotti, A., Yousef, S., Sputtek, A., Sturner, K. H., Stellmann, J. P., Breiden, P., Reinhardt, S., Schulze, C., Bester, M., Heesen, C., Schippling, S., Miller, S. D., Sospedra, M., Martin, R., 2013. Antigen-specific tolerance by autologous myelin peptide-coupled cells: a phase 1 trial in multiple sclerosis. Sci Transl Med 5, 188ra175.
McMahon, E. J., Bailey, S. L., Castenada, C. V., Waldner, H., Miller, S. D., 2005. Epitope spreading initiates in the CNS in two mouse models of multiple sclerosis. Nat Med 11, 335-339.
Miller, D. H., Chard, D. T., Ciccarelli, O., 2012. Clinically isolated syndromes. Lancet Neurol 11, 157-169.
Miller, D. H., Grossman, R. I., Reingold, S. C., McFarland, H. F., 1998. The role of magnetic resonance techniques in understanding and managing multiple sclerosis. Brain 121 (Pt 1), 3-24.
Miller, S. D., Katz-Levy, Y., Neville, K. L., Vanderlugt, C. L., 2001. Virus-induced autoimmunity: epitope spreading to myelin autoepitopes in Theiler's virus infection of the central nervous system. Adv Virus Res 56, 199-217.
Miller, S. D., McMahon, E. J., Schreiner, B., Bailey, S. L., 2007. Antigen presentation in the CNS by myeloid dendritic cells drives progression of relapsing experimental autoimmune encephalomyelitis. Ann N Y Acad Sci 1103, 179-191.
Nyakeriga, A. M., Garg, H., Joshi, A., 2012. TCR-Induced T cell activation leads to simultaneous phosphorylation at Y505 and Y394 of p56(lck) residues. Cytometry A.
O'Connor, K. C., Appel, H., Bregoli, L., Call, M. E., Catz, I., Chan, J. A., Moore, N. H., Warren, K. G., Wong, S. J., Hafler, D. A., Wucherpfennig, K. W., 2005. Antibodies from inflamed central nervous system tissue recognize myelin oligodendrocyte glycoprotein. J Immunol 175, 1974-1982.
Polman, C. H., Reingold, S. C., Banwell, B., Clanet, M., Cohen, J. A., Filippi, M., Fujihara, K., Havrdova, E., Hutchinson, M., Kappos, L., Lublin, F. D., Montalban, X., O'Connor, P., Sandberg-Wollheim, M., Thompson, A. J., Waubant, E., Weinshenker, B., Wolinsky, J. S., 2011. Diagnostic criteria for multiple sclerosis: 2010 revisions to the McDonald criteria. Ann Neurol 69, 292-302.
Polman, C. H., Wolinsky, J. S., Reingold, S. C., 2005. Multiple sclerosis diagnostic criteria: three years later. Mult Scler 11, 5-12.
Sawcer, S., 2008. The complex genetics of multiple sclerosis: pitfalls and prospects. Brain 131, 3118-3131.
Sawcer, S., et al., Genetic risk and a primary role for cell-mediated immune mechanisms in multiple sclerosis. Nature 476, 214-219.
Siebert, J. C., Inokuma, M., Waid, D. M., Pennock, N. D., Vaitaitis, G. M., Disis, M. L., Dunne, J. F., Wagner, D. H., Jr., Maecker, H. T., 2008. An analytical workflow for investigating cytokine profiles. Cytometry A 73, 289-298.
Smith, C. E., Miller, S. D., 2006. Multi-peptide coupled-cell tolerance ameliorates ongoing relapsing EAE associated with multiple pathogenic autoreactivities. J Autoimmun 27, 218-231.
Steck, A. K., Rewers, M. J., 2011. Genetics of type 1 diabetes. Clin Chem 57, 176-185.
Thorsby, E., Ronningen, K. S., 1993. Particular HLA-DQ molecules play a dominant role in determining susceptibility or resistance to type 1 (insulin-dependent) diabetes mellitus. Diabetologia 36, 371-377.
Vaitaitis, G., Wagner Jr., D. H., 2010. CD40 Glycoforms and TNF-Receptors 1 and 2 in the Formation of CD40 Receptor(s) in Autoimmunity. Mol. Immunol. 47, 2303-2313.
Vaitaitis, G., Waid, D. M., Wagner Jr., D., 2010. The Expanding Role of TNF-Receptor Super Family Member CD40 (tnfrsf5) in Autoimmune Disease: Focus on Th40 Cells Curr Immunol Rev 6, 130-137.
Vaitaitis, G. M., Carter, J. R., Waid, D. M., Olmstead, M. H., Wagner, D. H., Jr., 2013. An Alternative Role for Foxp3 As an Effector T Cell Regulator Controlled through CD40. J Immunol.
Vaitaitis, G. M., Poulin, M., Sanderson, R. J., Haskins, K., Wagner, D. H., Jr., 2003. Cutting edge: CD40-induced expression of recombination activating gene (RAG) 1 and RAG2: a mechanism for the generation of autoaggressive T cells in the periphery. J Immunol 170, 3455-3459.
Vaitaitis, G. M., Wagner, D. H., Jr., 2008. High distribution of CD40 and TRAF2 in Th40 T cell rafts leads to preferential survival of this auto-aggressive population in autoimmunity. PLoS One 3, e2076.
Vaitaitis, G. M., Wagner, D. H., Jr., 2010. CD40 glycoforms and TNF-receptors 1 and 2 in the formation of CD40 receptor(s) in autoimmunity. Mol Immunol 47, 2303-2313
Vaitaitis, G. M., Wagner, D. H., Jr., 2012. Galectin-9 controls CD40 signaling through a Tim-3 independent mechanism and redirects the cytokine profile of pathogenic T cells in autoimmunity. PLoS One 7, e38708.
Vaitaitis, G. M., Wagner Jr., D. H., 2013. CD40 interacts directly with RAG1 and RAG2 in autoaggressive T cells and Fas prevents CD40 induced RAG expression. Cell Mol Immunol In Press.
Wagner, D. H., Jr., Vaitaitis, G., Sanderson, R., Poulin, M., Dobbs, C., Haskins, K., 2002. Expression of CD40 identifies a unique pathogenic T cell population in type 1 diabetes. Proc Natl Acad Sci USA 99, 3782-3787.
Waid, D. M., Vaitaitis, G. M., Pennock, N. D., Wagner, D. H., Jr., 2008. Disruption of the homeostatic balance between autoaggressive (CD4+CD40+) and regulatory (CD4+CD25+FoxP3+) T cells promotes diabetes. J Leukoc Biol 84, 431-439.
Waid, D. M., Vaitaitis, G. M., Wagner, D. H., Jr., 2004. Peripheral CD4loCD40+ auto-aggressive T cell expansion during insulin-dependent diabetes mellitus. Eur J Immunol 34, 1488-1497.
Waid, D. M., Wagner, R. J., Putnam, A., Vaitaitis, G. M., Pennock, N. D., Calverley, D. C., Gottlieb, P., Wagner, D. H., Jr., 2007. A unique T cell subset described as CD4loCD40+ T cells (TCD40) in human type 1 diabetes. Clin Immunol 124, 138-148.
Wucherpfennig, K. W., Hafler, D. A., 1995. A review of T-cell receptors in multiple sclerosis: clonal expansion and persistence of human T-cells specific for an immunodominant myelin basic protein peptide. Ann N Y Acad Sci 756, 241-258.
Zhang, G. X., Yu, S., Gran, B., Li, J., Calida, D., Ventura, E., Chen, X., Rostami, A., 2004. T cell and antibody responses in remitting-relapsing experimental autoimmune encephalomyelitis in (C57BL/6×SJL) F1 mice. J Neuroimmunol 148, 1-10.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1-5. (canceled)

6. The method of claim 30, wherein the control sample is a sample from at least one subject known not to have multiple sclerosis.

7. The method of claim 30, wherein the control sample is a sample from at least one subject known to have multiple sclerosis.

8. The method of claim 30, wherein the control sample is a sample from at least one subject known to have relapsing-remitting multiple sclerosis (RRMS).

9. The method of claim 30, wherein the control sample is a sample from at least one subject known to have primary-progressive multiple sclerosis (PPMS).

10. The method of claim 30, wherein the control sample is a sample from at least one subject known to have secondary-progressive multiple sclerosis (SPMS).

11. The method of claim 30, wherein the control sample is a baseline sample obtained from the subject at an earlier date.

12. The method of claim 30, wherein the control sample is a sample obtained from at least one Type-1 Diabetes (T1D) patient.

13. The method of claim 30, wherein the control sample is a sample obtained from at least one Type-2 Diabetes (T2D) patient.

14. The method of claim 30, wherein the sample is whole blood, plasma, serum, or a subfraction of whole blood.

15. The method of claim 30, wherein the method further comprises determining the percentage of cells expressing additional markers selected from CD4, CD40, CD8, CD25, CD45, TCRV8.3+, CXCR3, and CCR5.

16. The method of claim 34, wherein the method comprises staining with a labeled antibody that specifically recognizes a protein selected from CD40, CD4, CD8, CD25 and CD45 and analyzing the stained cells by flow cytometry to determine the percentage of stained cells in the sample.

17. The method of claim 34, wherein the subject is being treated by the administration of an interferon.

18-25. (canceled)

26. A method for identifying and treating a patient for multiple sclerosis, which method comprises detecting the percentage of Th40 cells in T-cells in a blood sample from the patient and treating the patient with elevated percentage of Th40 cells relative to a percentage of Th40 cells in a blood sample from a normal control sample with a drug in an amount effective to decrease the percentage of Th40 cells in the patient's blood.

27. The method of claim 26, wherein the percentage of Th40 cells in the sample is assessed by staining with a labeled antibody that specifically recognizes a protein selected from CD40, CD4, CD8, CD25 and CD45 and analyzing the stained cells by flow cytometry to determine the percentage of stained cells in the sample.

28. The method of claim 26, further comprising administering a drug selected from at least one of Aubagio (teriflunomide), betaseron (interferon-b (type 1)), Avonex (Interferon-b-1b), Rebif (interferon-beta-1a), Copaxone (Glatiramer Acetate), Tysabri (Natalizumab), Novantrone (mitoxantrone), Gilenya (fingolimod), Tecfidera (dimethyl fumarate), and Lemtrada.

29. (canceled)

30. A method to confirm or rule out diagnosis of multiple sclerosis (MS) in a subject, comprising:
determining the percentage of Th40 cells in a sample isolated from a subject presenting with:
(i) typical clinically isolated syndrome (CIS) suggestive of MS, or
(ii) symptoms consistent with a CNS inflammatory demyelinating disease,
comparing the percentage of Th40 cells to a control sample or a standard value, and,
diagnosing multiple sclerosis in the subject having an increase in the percentage of Th40 cells in the sample from the subject relative to the control sample or standard value; or,
ruling out multiple sclerosis in the subject having no increase or a decrease in the percentage of Th40 cells in the sample from the subject relative to the control sample or standard value.

31. The method of claim 30, wherein the percentage of Th40 cells in the sample is assessed by staining with a labeled antibody that specifically recognizes a protein selected from CD40, CD4, CD8, CD25 and CD45 and analyzing the stained cells by flow cytometry to determine the percentage of stained cells in the sample.

32. (canceled)

33. (canceled)

34. A method to confirm or rule out the presence of relapsing/remitting multiple sclerosis (RRMS) in a subject, comprising:
determining the percentage of Th40 cells in a sample isolated from a subject presenting with a clinically isolated syndrome (CIS) suggestive of MS, and/or Radiologically Isolated Syndrome (RIS) suggestive of MS;
comparing the percentage of Th40 cells to a control sample or a standard value, and,
diagnosing RRMS in the subject having an increase in the percentage of Th40 cells in the sample from the subject relative to the control sample or standard value is indicative of multiple sclerosis in the subject; or,
ruling out a relapse of MS in the subject having no increase or a decrease in the percentage of Th40 cells in the sample from the subject relative to the control sample or standard value is indicative of multiple sclerosis in the subject.

35. (canceled)
